(19) United States
(12) Patent Application Publication
(10) Pub. No.: US 2004/0122033 A1
(43) Pub. Date: Jun. 24, 2004

(54) Combination therapy for the treatment of obesity

(76) Inventors: Ravi P. Nargund, East Brunswick, NJ (US); Leonardus H.T. Van Der Ploeg, Scotch Plains, NJ (US); Tung M. Fong, Somerset, NJ (US); Douglas J. MacNeil, Westfield, NJ (US); Howard Y. Chen, Westfield, NJ (US); Donald J. Marsh, Hillsborough, NJ (US); Jeffrey Warmke, Edison, NJ (US)

Correspondence Address: MERCK AND CO INC, P O BOX 2000, RAHWAY, NJ, 070650907

(21) Appl. No.: 10/730,704

(22) Filed: Dec. 8, 2003

Related U.S. Application Data
(60) Provisional application No. 60/432,063, filed on Dec. 10, 2002.

(51) Int. Cl. A61K 31/485; A61K 31/19

(52) U.S. Cl. 514/282; 514/571

(57) ABSTRACT

The present invention relates to compositions comprising an appetite suppressant and/or a metabolic rate enhancer and/or a nutrient absorption inhibitor useful for the treatment of obesity, and obesity-related disorders. The present invention further relates to methods of treating or preventing obesity, and obesity-related disorders, in a subject in need thereof by administering a composition of the present invention. The present invention further provides for pharmaceutical compositions, medicaments, and kits useful in carrying out these methods.

### BACKGROUND OF THE INVENTION

Obesity, which can be defined as a body weight more than 20% above the ideal body weight, is a major health concern in Western societies. It is estimated that about 97 million adults in the United States are overweight or obese. Obesity is the result of a positive energy balance, as a consequence of increased ratio of caloric intake to energy expenditure. The molecular factors regulating food intake and body weight balance are incompletely understood. [B. Staels et al., J. Biol. Chem. 270(27), 15958 (1995); F. Lonnquist et al., Nature Medicine 1(9), 950 (1995)]. Although the genetic and/or environmental factors leading to obesity are poorly understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Obesity is often treated by encouraging patients to lose weight by reducing their food intake or by increasing their exercise level and therefore increasing their energy output. A sustained weight loss of 5% to 10% of body weight has been shown to improve the co-morbidities associated with obesity, such as diabetes and hypertension, and can lead to improvement of obesity-related conditions such as osteoarthritis, sleep apnea and pulmonary and cardiac dysfunction.

Weight loss drugs that are currently used in monotherapy for the treatment of obesity have limited efficacy and significant side effects. Studies of the weight loss medications orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. In particular, both sibutramine and orlistat reduce body weight less than 10% over a 6 month or a 1 year period. Preclinical studies have also found that most agents, such as sibutramine, fenfluramine, Y5 antagonists, CB-1 inverse agonists and Mc4r agonists, potently inhibit food intake and decrease body weight initially. However, during chronic treatment periods of greater than 10 days the efficacy of these agents decreases yielding no more than 10% body weight loss compared to control. Obese humans can easily mass over 150 kg and would, therefore, need to lose more than 50% of their body mass to return to a normal body mass. For these patients, single agents are likely to have minimal therapeutic utility. The side effects of these drugs and anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

While single agents may be efficacious for the treatment of obesity in certain patients, due to the polygenic nature of obesity etiology, it is predicted that no single agent will be efficacious for the vast majority of obese patients. Combination therapy is more likely to achieve the desired medical benefits without the trial and error involved in prescribing each agent individually during primary care.

Commercially available combination therapies, which include phentermine as one of the components, have lead to mixed results. Phentermine was prescribed with fenfluramine (Pondimin®) or dexfenfluramine (Redux®) as a combination therapy known as fen-phen, which was withdrawn from the market in 1997 based on studies suggesting that the drugs cause damage to the mitral valve of the heart and pulmonary hypertension. Additionally, both fenfluramine and phentamine work through the same biological mechanism, namely the serotonin and norepinephrine pathway. Due to the side effects and limited efficacy of the anti-obesity drugs currently available for mono-and combination therapy, there is a need for a combination weight loss treatment with enhanced efficacy and fewer undesirable side effects. The instant invention addresses this problem by providing a combination therapy, useful to treat obesity and obesity-related disorders, comprised of two agents with different biological mechanisms of action, thereby reducing the probability of undesirable side effects while enhancing efficacy.

It has now been found that agents that work through one specific mode of action, or via one specific physiological pathway, are still efficacious when a second pathway, known to be involved in energy homeostasis in rodents, is absent. The combination of two agents that affect energy homeostasis based on different biological mechanisms of action is advantageous in the treatment of obesity, and obesity-related disorders, over the treatment with two agents that work through the same pathway, or the treatment with either agent alone.

As a result, combination therapies with agents that work via different biological mechanisms of action are more effective than currently available monotherapies and combination therapies, which are based on one agent or on agents that work via the same mode of action.

Based on this finding, a combination of two appetite suppressants with different biological mechanisms of action, a combination of two metabolic rate enhancers with different biological mechanisms of action, and a combination of two nutrient absorption inhibitors with different biological mechanisms of action is advantageous in the treatment of obesity, and obesity-related disorders, over the treatment with two agents that work via the same mode of action or the treatment by the individual agents alone. Furthermore, combinations of an appetite suppressant and/or a metabolic rate enhancer and/or a nutrient absorption inhibitor, with different biological mechanisms of action, are advantageous in the treatment of obesity, and obesity-related disorders, over the treatment with two agents that work via the same mode of action or the treatment by the individual agents alone.

It is an object of the present invention to identify compositions comprising appetite suppressants, and/or metabolic rate enhancers, and/or nutrient absorption inhibitors useful for the treatment of obesity, and obesity-related disorders. It is another object of the invention to identify methods of treating obesity, and obesity-related disorders. It is yet another object of the invention to identify methods of preventing obesity, and obesity-related disorders.

It is a further object of the present invention to provide pharmaceutical compositions comprising appetite suppressants and/or metabolic rate enhancers and/or nutrient absorption inhibitors. It is yet a further object of the present invention to provide a method of manufacture of a medicament useful in the treatment of obesity, and obesity-related disorders.

### SUMMARY OF THE INVENTION

The present invention provides compositions comprising an appetite suppressant and/or a metabolic rate enhancer and/or a nutrient absorption inhibitor useful in the treatment or prevention of obesity, and obesity-related disorders.

The present invention further provides compositions comprising two appetite suppressants with different biological mechanisms of action, or two metabolic rate enhancers with different biological mechanisms of action, or two nutrient absorption inhibitors with different biological mechanisms of action useful in the treatment and prevention of obesity, and obesity-related disorders.

The present invention provides compositions comprising an appetite suppressant selected from the group consisting of: a 5HT (serotonin) transporter inhibitor, a NE (norepinephrine) transporter inhibitor, a CB-1 (cannabinoind-1 receptor) antagonist/inverse agonist, a ghrelin antagonist, a H3 (histamine H3) antagonist/inverse agonist, a MCH1R (melanin concentrating hormone 1R) antagonist, a MCH2R (melanin concentrating hormone 2R) agonist/antagonist, a NPY1 (neuropeptide Y Y1) antagonist, a NPY5 (neuropeptide Y Y5) antagonist, a NPY2 (neuropeptide Y Y2) agonist, NPY4 (neuropeptide Y Y4) agonist, a mGluR5 (metabotropic glutamate subtype 5 receptor) antagonist; leptin, a leptin derivative, an opioid antagonist, an orexin antagonist, a BRS3 (bombesin receptor subtype 3) agonist, a CCK-A (cholecystokinin-A) agonist, CNTF (ciliary neurotrophic factor), a CNTF derivative, a 5HT2c (serotonin receptor 2c) agonist, a Mc4r (melanocortin-4 receptor) agonist, a monoamine reuptake inhibitor, a serotonin reuptake inhibitor, a GLP-1 (glucagon-like peptide 1) agonist, topiramate, and phytopharm compound 57;and pharmaceutically acceptable salts and esters thereof.

The present invention provides compositions comprising a metabolic rate enhancer selected from the group consisting of: an ACC2 (acetyl-CoA carboxylase-2) inhibitor, a β3 (beta adrenergic receptor 3) agonist, a DGAT1 (diacylglycerol acyltransferase 1) inhibitor, a DGAT2 (diacylglycerol acyltransferase 2)inhibitor, a FAS (fatty acid synthase) inhibitor, a PDE (phosphodiesterase) inhibitor, a thyroid hormone β agonist, an UCP-1 (uncoupling protein 1), 2, or 3 activator, an acyl-estrogen, a glucocorticoid antagonist, an 11β HSD-1 (11-beta hydroxy steroid dehydrogenase type 1) inhibitor, a Mc3r (melanocortin-3 receptor) agonist, and a SCD-1 (stearoyl-CoA desaturase-1); and pharmaceutically acceptable salts and esters thereof.

The present invention provides compositions comprising a nutrient absorption inhibitor selected from the group consisting of: a lipase inhibitor, a fatty acid transporter inhibitor, a dicarboxylate transporter inhibitor, a glucose transporter inhibitor, a phosphate transporter inhibitor; and pharmaceutically acceptable salts and esters thereof.

The compositions of the present invention are useful in the treatment or prevention of obesity and the following obesity-related disorders: overeating; bulimia; hypertension; diabetes, elevated plasma insulin concentrations; insulin resistance; dyslipidemias; hyperlipidemia; endometrial, breast, prostate and colon cancer; osteoarthritis; obstructive sleep apnea; cholelithiasis; gallstones; abnormal heart rhythms; heart arrythmias; myocardial infarction; congestive heart failure; coronary heart disease; sudden death; stroke; polycystic ovary disease; craniopharyngioma; the Prader-Willi Syndrome; Frohlich's syndrome; GH-deficient subjects; normal variant short stature; Turner's syndrome; metabolic syndrome; and other pathological conditions showing reduced metabolic activity or a decrease in resting energy expenditure as a percentage of total fat-free mass, e.g, children with acute lymphoblastic leukemia.

The present invention is also concerned with treatment of these conditions, and the use of the compositions of the present invention for manufacture of a medicament useful for treating these conditions.

The invention is also concerned with pharmaceutical compositions comprising an appetite suppressant and/or a metabolic rate enhancer and/or a nutrient absorption inhibitor, as active ingredients. The invention is further concerned with pharmaceutical compositions comprising two appetite suppressants with different biological mechanisms of action, or two metabolic rate enhancers with different biological mechanisms of action, or two nutrient absorption inhibitors with different biological mechanisms of action, as active ingredients.

The present invention is also concerned with the use of an appetite suppressant and/or a metabolic rate enhancer and/or a nutrient absorption inhibitor, for the manufacture of a medicament for the treatment of obesity, and obesity-related disorders, which comprises an effective amount of the appetite suppressant and/or the metabolic rate enhancer and/or the nutrient absorption inhibitor, together or separately. The present invention is further concerned with the use of two appetite suppressants, or two metabolic rate enhancers, or two nutrient absorption inhibitors, for the manufacture of a medicament for the treatment of obesity, and obesity-related disorders, which comprises an effective amount of the appetite suppressant and/or the metabolic rate enhancer and/or the nutrient absorption inhibitor, together or separately.

The present invention is also concerned with a product containing an appetite suppressant and/or a metabolic rate enhancer and/or a nutrient absorption inhibitor, as a combined preparation for simultaneous, separate or sequential use in obesity, and obesity related disorders. The present invention is also concerned with a product containing two appetite suppressants with different biological mechanisms of action, or two metabolic rate enhancers with different biological mechanisms of action, or two nutrient absorption inhibitors with different biological mechanisms of action, as a combined preparation for simultaneous, separate or sequential use in obesity, and obesity-related disorders.

The present invention also relates to the treatment of obesity, and obesity-related disorders, with a combination of an appetite suppressant and/or a metabolic rate enhancer and/or a nutrient absorption inhibitor, which may be administered separately. The present invention further relates to the treatment of obesity, and obesity-related disorders, with a combination of two appetite suppressants with different biological mechanisms of action, or two metabolic rate enhancers with different biological mechanisms of action, or two nutrient absorption inhibitors with different biological mechanisms of action, which may be administered separately. The invention also relates to combining separate pharmaceutical combinations into a kit form.

### BRIEF DESCRIPTION OF THE DRAWINGS

FIG. 1. Shows the effect on overnight food intake of wild type mice, Mch1r+/+ (left), treated with either aCSF, artificial cerebrospinal fluid (solid), or with 1 μg of MT-II, a melanocortin receptor agonist (patterned), or MCH-1R knock out mice, Mch1r−/− (right), treated with aCSF (solid) or treated with 1 μg of MT-II (patterned).

FIG. 2. Shows the overnight change in bodyweight of wild type mice, Mch1r+/+ (left), treated with either aCSF, artificial cerebrospinal fluid (solid), or with 1 μg of MT-II, a melanocortin receptor agonist (patterned), or MCH-1R knock out mice, Mch1r−/− (right), treated with aCSF (solid) or with 1 μg of MT-II (patterned).

FIG. 3. Shows the effect on overnight food intake of wild type mice (AGRP+/+NPY+/+, WT), NPY knock out mice (NPY−/−, NPY KO) and AGRP-NPY double knock out mice (AGRP−/−NPY−/−, agouti protein knockout and neuropeptide Y knockout mice) treated with 3 mg/kg PO of AM 251, a CB-1 inverse agonist.

FIG. 4. Shows the effect of the CB1R inverse agonist AM251 (3 mpk) on 2 hour and overnight food intake of wild type (Mch1r+/+) or MCH1R KO (Mch1r−/−) mice.

### DETAILED DESCRIPTION OF THE INVENTION

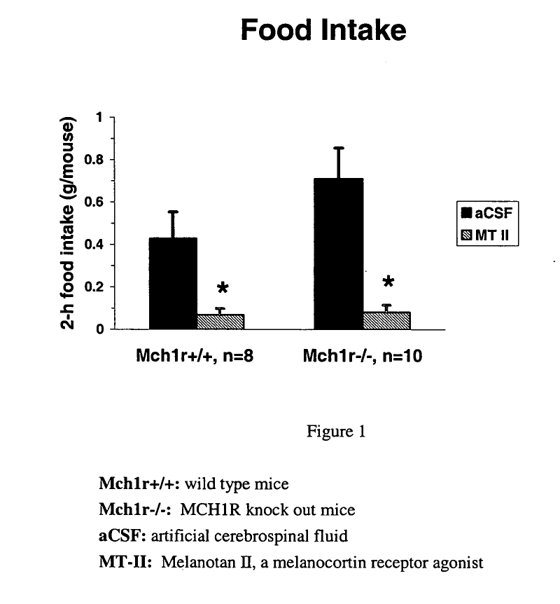

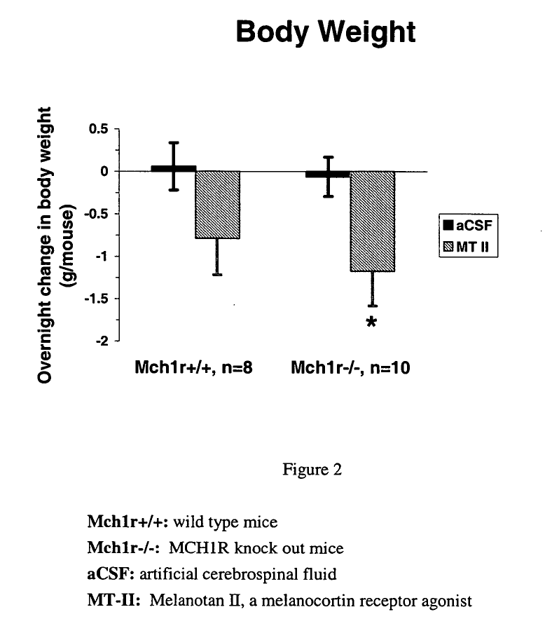

Recently, it was surprisingly found that the treatment of MCH-1R knock out mice with MT-II, a melanocortin receptor agonist, decreases overnight food intake by greater than 80%. This finding is supported by data showing that there was a significantly greater decrease in food intake in MCH-1R knock out mice treated with MT-II than in wild type mice treated with MT-II (See FIG. 1). This study also showed that there is a greater overnight decrease in bodyweight in MCH-1R knock out mice treated with MT-II than in wild type mice treated with MT-II (See FIG. 2). The study findings also surprisingly showed that MCH1R knock out mice eat more than the wild type mice and that treating MCH1R knock out mice with MT-II, a melanocortin agonist, still results in food intake inhibition. These studies suggest that a melanocortin receptor agonist will be efficacious in the presence of a MCH receptor antagonist. As a result, a combination of a melanocortin receptor agonist and a MCH receptor antagonist is surprisingly more efficacious, in the treatment of obesity, and obesity-related disorders, than treatment with either agent alone. These studies further suggest that combinations of two agents that operate based on parallel biological mechanisms are more efficacious than combinations of two agents that operate based on sequential biological mechanisms.

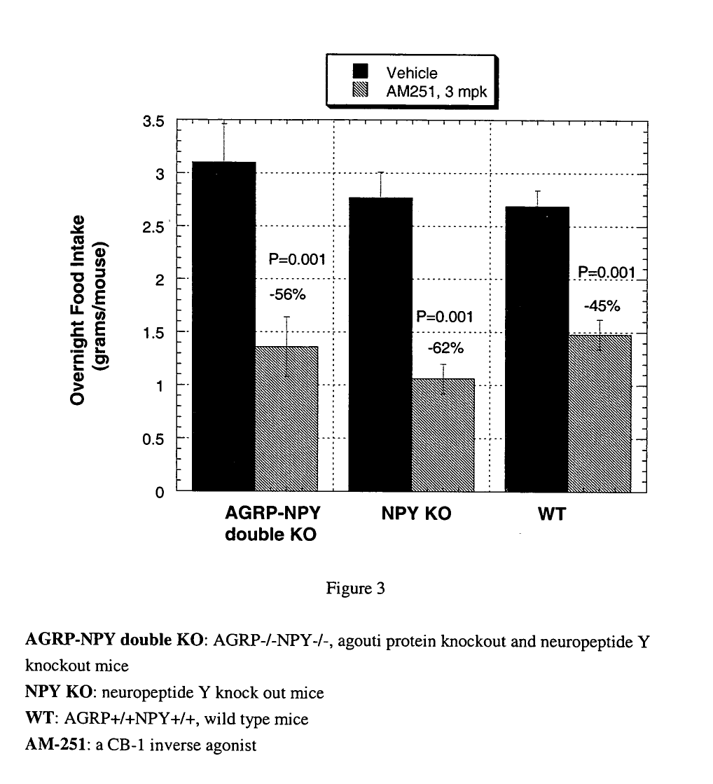

Another study showed that AM251, a CB1R inverse agonist, significantly inhibits the overnight food intake in wild type mice, in AGRP-NPY (agouti protein, neuropeptide Y) double knock out mice and in NPY knock out mice (See FIG. 3). AGRP is an endogenous Mc4r antagonist that enhances food intake; and NPY is also known to enhance food intake. This study suggests that a CB-1 inverse agonist/antagonist will be efficacious even in the presence of a NPY antagonist, and that a CB-1 inverse agonist/antagonist will be efficacious in the presence of both a NPY antagonist and Mc4r agonist. The combination of a CB-1 inverse agonist/antagonist and a NPY antagonist has enhanced efficacy in the treatment of obesity and obesity-related diseases. Furthermore, the combination of a CB-1 inverse agonist/antagonist, a NPY antagonist and a Mc4r agonist also has enhanced efficacy in the treatment of obesity and obesity related diseases.

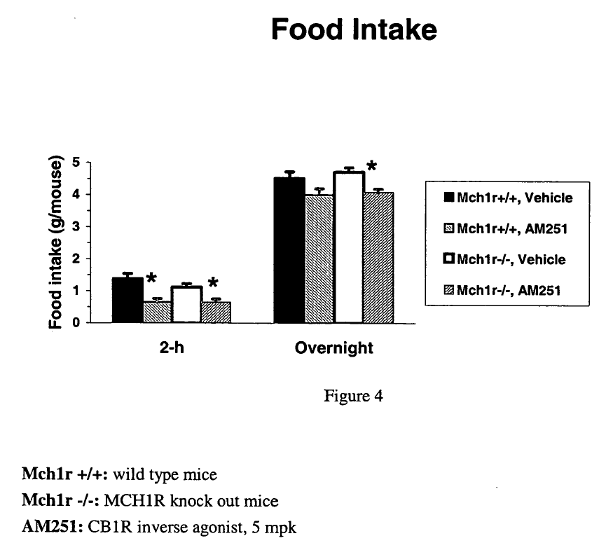

A further study showed that treatment of wild type mice and MCH1R knock out mice with AM251, a CB1 inverse agonist, inhibits the overnight food intake in both wild type and MCH-1R knock out mice (see FIG. 4). This study suggests that a CB-1 inverse agonist/antagonist will be efficacious even in the presence of a MCH-1 receptor antagonist. This study further suggests that the combination of a CB-1 inverse agonist/antagonist and a MCH receptor antagonist has enhanced efficacy in the treatment of obesity and obesity-related disorders.

Based on these studies, a combination treatment with two agents, that work via different biological mechanisms is expected to result in greater decrease in food intake, and greater weight loss, than treatment with either agent alone.

These studies also show that agents that work through one specific physiological mode of action, or via one specific pathway, are still efficacious when a different physiological pathway, which is known to be involved in energy homeostasis in rodents, is absent.

Based on these findings, the combination therapy with two agents that affect energy homeostasis via different biological mechanisms of action is surprisingly more efficacious, in the treatment of obesity, and obesity-related disorders, than the treatment with two agents that work through the same mechanistic pathway, or the treatment with the individual agents alone. More particularly, combining appetite suppressants and/or metabolic rate enhancers and/or nutrient absorption inhibitors, whose biological mechanism of action differs, is advantageous in the treatment of obesity over the treatment of two agents that work through the same biological mechanism of action. Combining two agents with different biological mechanisms of action at clinical or sub-clinical doses will produce clinical efficacy without the side-effects of treatment with either agent alone at the monotherapy clinical dose.

The present invention provides compositions comprising at least one appetite suppressant and/or at least one metabolic rate enhancer and/or at least one nutrient absorption inhibitors useful in the treatment or prevention of obesity, and obesity-related disorders.

The methods and compositions of the present invention comprise an appetite suppressant. The appetite suppressant useful in the compositions of the present invention may be any agent useful to decrease food intake known in the art. The appetite suppressant may be peptidal or non-peptidal in nature; however, the use of a non-peptidal agent is preferred. For convenience, the use of an orally active appetite suppressant is also preferred. The appetite suppressant useful in the compositions of the present invention is selected from the group consisting of:

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY5 antagonist,

(10) a NPY2 agonist,

(11) a NPY4 agonist,

(12) a mGluR5 antagonist,

(13) leptin,

(14) a leptin agonist/modulator,

(15) a leptin derivative,

(16) an opioid antagonist,

(17) an orexin antagonist,

(18) a BRS3 agonist,

(19) a CCK-A agonist,

(20) CNTF,

(21) a CNTF derivative,

(22) a CNTF agonist/modulator,

(23) 5HT2c agonist,

(24) a Mc4r agonist,

(25) a monoamine reuptake inhibitor,

(26) a serotonin reuptake inhibitor,

(27) a GLP-1 agonist,

(28) axokine,

(29) fenfluramine,

(30) nalmafene,

(31) phentermine,

(32) rimonabant,

(33) sibutramine,

(34) topiramate, and

(35) phytopharm compound 57;

and pharmaceutically acceptable salts and esters thereof.

In one embodiment of the present invention, the appetite suppressant is selected from the group consisting of:

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) leptin,

(13) a leptin agonist/modulator,

(14) a leptin derivative,

(15) an opioid antagonist,

(16) an orexin antagonist,

(17) a BRS3 agonist,

(18) a CCK-A agonist,

(19) CNTF,

(20) a CNTF agonist/modulator,

(21) a CNTF derivative,

(22) a 5HT2c agonist,

(23) a Mc4r agonist,

(24) a monoamine reuptake inhibitor,

(25) a serotonin reuptake inhibitor,

(26) a GLP-1 agonist,

(27) axokine,

(28) fenfluramine,

(29) nalmafene,

(30) phentermine,

(31) rimonabant,

(32) sibutramine,

(33) topiramate, and

(34) phytopharm compound 57;

and pharmaceutically acceptable salts and esters thereof.

In a class of this embodiment, the appetite suppressant is selected from the group consisting of:

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) an opioid antagonist,

(13) an orexin antagonist,

(14) a BRS3 agonist,

(15) a CCK-A agonist,

(16) CNTF,

(17) a CNTF agonist/modulator,

(18) a CNTF derivative,

(19) a 5HT2c agonist,

(20) a Mc4r agonist,

(21) a monoamine reuptake inhibitor,

(22) a serotonin reuptake inhibitor,

(23) a GLP-1 agonist,

(24) axokine,

(25) fenfluramine,

(26) nalmafene,

(27) phentermine,

(28) rimonabant,

(29) sibutramine, and

(30) topiramate;

and pharmaceutically acceptable salts and esters thereof.

In another class of this embodiment, the appetite suppressant is selected from the group consisting of:

(1) a 5HT transporter inhibitor,

(2) a NE transporter inhibitor,

(3) a ghrelin antagonist,

(4) a H3 antagonist/inverse agonist,

(5) a MCH1R antagonist,

(6) a MCH2R agonist/antagonist,

(7) an opioid antagonist,

(8) an orexin antagonist,

(9) a BRS3 agonist,

(10) a CCK-A agonist,

(1 1) CNTF,

(12) a CNTF agonist/modulator,

(13) a CNTF derivative,

(14) a 5HT2c agonist,

(15) a Mc4r agonist,

(16) a monoamine reuptake inhibitor,

(17) a serotonin reuptake inhibitor,

(18) a GLP-1 agonist,

(19) axokine,

(20) fenfluramine,

(21) nalmafene,

(22) phentermine,

(23) rimonabant,

(24) sibutramine,

(25) topiramate, and

(26) phytopharm compound 57;

and pharmaceutically acceptable salts and esters thereof.

In a subclass of this class, the appetite suppressant is selected from the group consisting of:

(1) a 5HT transporter inhibitor,

(2) a NE transporter inhibitor,

(3) a ghrelin antagonist,

(4) a H3 antagonist/inverse agonist,

(5) a MCH1R antagonist,

(6) a MCH2R agonist/antagonist,

(7) an opioid antagonist,

(8) an orexin antagonist,

(9) a BRS3 agonist,

(10) a CCK-A agonist,

(11) CNTF,

(12) a CNTF derivative,

(13) a Mc4r agonist,

(14) a monoamine reuptake inhibitor, and

(15) a serotonin reuptake inhibitor;

and pharmaceutically acceptable salts and esters thereof.

In another subclass of this class, the appetite suppressant is selected from the group consisting of:

(1) a 5HT transporter inhibitor,

(2) a NE transporter inhibitor,

(3) a ghrelin antagonist,

(4) a H3 antagonist/inverse agonist,

(5) a MCH2R agonist,

(6) an opioid antagonist,

(7) an orexin antagonist,

(8) a BRS3 agonist,

(9) a CCK-A agonist,

(10) CNTF, and

(11) a CNTF derivative,

and pharmaceutically acceptable salts and esters thereof.

In another class of this embodiment, the appetite suppressant is a 5HT transporter inhibitor, and pharmaceutically acceptable salts or esters thereof. In another class of this embodiment, the appetite suppressant is a NE transporter inhibitor, and pharmaceutically acceptable salts or esters thereof. In another class of this embodiment, the appetite suppressant is a CB-1 antagonist/inverse agonist, and pharmaceutically acceptable salts or esters thereof. In a subclass of this class, the CB-1 antagonist/inverse agonist is selected from rimonabant, and pharmaceutically acceptable salts or esters thereof. In another class of this embodiment, the appetite suppressant is a ghrelin antagonist, and pharmaceutically acceptable salts or esters thereof. In another class of this embodiment, the appetite suppressant is a H3 antagonist/inverse agonist, and pharmaceutically acceptable salts or esters thereof. In another class of this embodiment, the appetite suppressant is a MCH1R antagonist, and pharmaceutically acceptable salts or esters thereof. In another class of this embodiment, the appetite suppressant is a MCH2R agonist/antagonist, and pharmaceutically acceptable salts or esters thereof.

In another class of this embodiment, the appetite suppressant is a NPY1 antagonist, and pharmaceutically acceptable salts or esters thereof. In another class of this embodiment, the appetite suppressant is leptin, and pharmaceutically acceptable salts or esters thereof. In another class of this embodiment, the appetite suppressant is a leptin derivative, and pharmaceutically acceptable salts or esters thereof. In another class of this embodiment, the appetite suppressant is an opioid antagonist, and pharmaceutically acceptable salts or esters thereof. In a subclass of this class, the opioid antagonist is selected from nalmefene, and pharmaceutically acceptable salts or esters thereof. In another class of this embodiment, the appetite suppressant is an orexin antagonist, and pharmaceutically acceptable salts or esters thereof. In another class of this embodiment, the appetite suppressant is a BRS3 agonist, and pharmaceutically acceptable salts or esters thereof. In another class of this embodiment, the appetite suppressant is a CCK-A agonist, and pharmaceutically acceptable salts or esters thereof.

In another class of this embodiment, the appetite suppressant is CNTF, and pharmaceutically acceptable salts or esters thereof. In another class of this embodiment, the appetite suppressant is a CNTF derivative, and pharmaceutically acceptable salts or esters thereof. In subclass of this class, the CNTF derivative is selected from axokine, and pharmaceutically acceptable salts or esters thereof. In another class of this embodiment, the appetite suppressant is a 5HT2c agonist, and pharmaceutically acceptable salts or esters thereof. In another class of this embodiment, the appetite suppressant is a Mc4r agonist, and pharmaceutically acceptable salts or esters thereof.

In another class of this embodiment, the appetite suppressant is a monoamine reuptake inhibitor, and pharmaceutically acceptable salts or esters thereof. In a subclass of this class, the monoamine reuptake inhibitor is selected from sibutramine, and pharmaceutically acceptable salts and esters thereof. In another class of this embodiment, the appetite suppressant is a serotonin reuptake inhibitor, and pharmaceutically acceptable salts or esters thereof. In another class of this embodiment, the appetite suppressant is a GLP-1 agonist, and pharmaceutically acceptable salts or esters thereof. In another class of this embodiment, the appetite suppressant is topiramate, and pharmaceutically acceptable salts or esters thereof. In another class of this embodiment, the appetite suppressant is phytopharm compound 57, and pharmaceutically acceptable salts or esters thereof.

The methods and compositions of the present invention comprise a metabolic rate enhancer. The metabolic rate enhancer useful in the compositions of the present invention may be any agent useful to enhance metabolic rate known in the art. The metabolic rate enhancer may be peptidal or non-peptidal in nature; however, the use of a non-peptidal agent is preferred. For convenience, the use of an orally active metabolic rate enhancer is also preferred. The metabolic rate enhancer useful in the compositions of the present invention is selected from the group consisting of:

(1) an ACC2 inhibitor,

(2) a β3 agonist,

(3) a DGAT1 inhibitor,

(4) a DGAT2 inhibitor,

(5) a FAS inhibitor,

(6) a PDE inhibitor,

(7) a thyroid hormone β agonist,

(8) an UCP-1, 2, or 3 activator,

(9) an acyl-estrogen,

(10) a glucocorticoid antagonist,

(11) an 11β HSD-1 inhibitor;

(12) a Mc3r agonist;

(13) a SCD-1; and

(14) oleoyl-estrone,

and pharmaceutically acceptable salts and esters thereof.

In one embodiment of the present invention, the metabolic rate enhancer is selected from the group consisting of:

(1) an ACC2 inhibitor,

(2) a DGAT1 inhibitor,

(3) a DGAT2 inhibitor,

(4) a FAS inhibitor,

(5) a PDE inhibitor,

(6) a thyroid hormone β agonist,

(7) an UCP-1, 2, or 3 activator,

(8) an acyl-estrogen,

(9) a glucocorticoid antagonist,

(10) an 11β HSD-1 inhibitor; and

(11) oleoyl-estrone;

and pharmaceutically acceptable salts and esters thereof.

In another class of this embodiment, the metabolic rate enhancer is an ACC2 inhibitor, and pharmaceutically acceptable salts or esters thereof. In another class of this embodiment, the metabolic rate enhancer is a DGAT1 inhibitor, and pharmaceutically acceptable salts or esters thereof. In another class of this embodiment, the metabolic rate enhancer is a DGAT2 inhibitor, and pharmaceutically acceptable salts or esters thereof.

In another class of this embodiment, the metabolic rate enhancer is a FAS inhibitor, and pharmaceutically acceptable salts or esters thereof. In another class of this embodiment, the metabolic rate enhancer is a PDE inhibitor, and pharmaceutically acceptable salts or esters thereof. In another class of this embodiment, the metabolic rate enhancer is a thyroid hormone β agonist, and pharmaceutically acceptable salts or esters thereof. In another class of this embodiment, the metabolic rate enhancer is an UCP-1, 2, or 3 activator, and pharmaceutically acceptable salts or esters thereof. In another class of this embodiment, the metabolic rate enhancer is a glucocorticoid antagonist, and pharmaceutically acceptable salts or esters thereof. In another class of this embodiment, the metabolic rate enhancer is an acyl-estrogen, and pharmaceutically acceptable salts or esters thereof. In a subclass of this class, the acyl-estrogen, is selected from oleoyl-estrone, and pharmaceutically acceptable salts or esters thereof.

In another class of this embodiment, the metabolic rate enhancer is an 11β HSD-1 inhibitor, and pharmaceutically acceptable salts or esters thereof. In a subclass of this class, the 11β HSD-1 inhibitor is selected from the group consisting of

(1) 3-[(3,5,7-trimethyl-1-adamantyl)methyl]-6,7,8,9-tetrahydro-5H-[1,2,4]triazolo[4,3-a]azepine,

(2) 3-(1-adamantyl)-4-ethyl-5-(ethylthio)-4H-1,2,4-triazole,

(3) 3-adamantanyl-4,5,6,7,8,9,10,11,12,3a-decahydro-1,2,4-triazolo[4,3-a][11]annulene, and

(4) 3-(1-adamantyl)-5-(3,4,5-trimethoxyphenyl)-4-methyl-4H-1,2,4-triazole;

and pharmaceutically acceptable salts and esters thereof.

In another class of this embodiment, the metabolic rate enhancer is a Mc3r agonist, and pharmaceutically acceptable salts or esters thereof. In yet another class of this embodiment, the metabolic rate enhancer is a SCD-1, and pharmaceutically acceptable salts or esters thereof.

The methods and compositions of the present invention comprise a nutrient absorption inhibitor. The nutrient absorption inhibitor useful in the compositions of the present invention may be any nutrient absorption inhibitor known in the art. The nutrient absorption inhibitor may be peptidal or non-peptidal in nature, however, the use of a non-peptidal agent is preferred. For convenience, the use of an orally active nutrient absorption inhibitor is also preferred. The nutrient absorption inhibitor useful in the compositions of the present invention is selected from the group consisting of:

(1) a lipase inhibitor,

(2) a fatty acid transporter inhibitor,

(3) a dicarboxylate transporter inhibitor,

(4) a glucose transporter inhibitor,

(5) a phosphate transporter inhibitor, and

(6) orlistat,

and pharmaceutically acceptable salts and esters thereof.

In one embodiment of the present invention, the nutrient absorption inhibitor is selected from the group consisting of:

(1) a lipase inhibitor,

(2) a fatty acid transporter inhibitor,

(3) a dicarboxylate transporter inhibitor,

(4) a glucose transporter inhibitor, and

(5) orlistat,

and pharmaceutically acceptable salts and esters thereof.

In a class of this embodiment, the nutrient absorption inhibitor is a lipase inhibitor, and pharmaceutically acceptable salts or esters thereof. In a subclass of this embodiment, the lipase inhibitor is orlistat, and the pharmaceutically acceptable salts thereof. In another class of this embodiment, the nutrient absorption inhibitor is a fatty acid transporter inhibitor, and pharmaceutically acceptable salts or esters thereof. In another class of this embodiment, the nutrient absorption inhibitor is a dicarboxylate transporter inhibitor, and pharmaceutically acceptable salts or esters thereof. In another class of this embodiment, the nutrient absorption inhibitor is a glucose transporter inhibitor, and pharmaceutically acceptable salts or esters thereof.

In another embodiment of this invention, the nutrient absorption inhibitor is a phosphate transporter inhibitor, and pharmaceutically acceptable salts or esters thereof.

In another embodiment of the present invention, the composition comprises two appetite suppressants, and pharmaceutically acceptable salts and esters thereof, provided that the appetite suppressants have different biological mechanisms of action.

In class of this embodiment, the composition comprises two appetite suppressants, wherein each appetite suppressant is selected from the group consisting of:

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY5 antagonist,

(10) a NPY2 agonist,

(11) a NPY4 agonist,

(12) a mGluR5 antagonist,

(13) leptin,

(14) a leptin agonist/modulator,

(15) a leptin derivative,

(16) an opioid antagonist,

(17) an orexin antagonist,

(18) a BRS3 agonist,

(19) a CCK-A agonist,

(20) CNTF,

(21) a CNTF agonist/modulator,

(22) a CNTF derivative,

(23) a 5HT2c agonist,

(24) a Mc4r agonist,

(25) a monoamine reuptake inhibitor,

(26) a serotonin reuptake inhibitor,

(27) a GLP-1 agonist,

(28) axokine,

(29) fenfluramine,

(30) nalmafene,

(31) phentermine,

(32) rimonabant,

(33) sibutramine,

(34) topiramate, and

(35) phytopharm compound 57;

and pharmaceutically acceptable salts and esters thereof;

provided that when the first appetite suppressant is a NPY1 antagonist, then the second appetite suppressant is not selected from the group consisting of: a MCH1R antagonist, a MCH2R antagonist, a NPY5 antagonist, leptin, a leptin derivative, 5HT2c agonist, a Mc4r agonist, a serotonin reuptake inhibitor, and a GLP-1 agonist;

provided that when the first appetite suppressant is leptin, then the second appetite suppressant is not selected from the group consisting of: a MCH-1R antagonist, a MCH-2R antagonist, a NPY1 antagonist, a NPY5 antagonist, a leptin derivative, 5HT2c agonist, a Mc4r agonist, a serotonin reuptake inhibitor, a GLP-1 agonist, a CCK-A agonist, an opioid antagonist, and a monoamine reuptake inhibitor;

provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor;

provided that when the first appetite suppressant is an opioid antagonist, then the second appetite suppressant is not a serotonin reuptake inhibitor; and

provided that the appetite suppressants have different biological mechanisms of action.

In a subclass of this class, the appetite suppressant is selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) leptin,

(13) a leptin agonist/modulator,

(14) a leptin derivative,

(15) an opioid antagonist,

(16) an orexin antagonist,

(17) a BRS3 agonist,

(18) a CCK-A agonist,

(19) CNTF,

(20) a CNTF agonist/modulator,

(21) a CNTF derivative,

(22) a 5HT2c agonist,

(23) a Mc4r agonist,

(24) a monoamine reuptake inhibitor,

(25) a serotonin reuptake inhibitor,

(26) a GLP-1 agonist,

(27) axokine,

(28) fenfluramine,

(29) nalmafene,

(30) phentermine,

(31) rimonabant,

(32) sibutramine,

(33) topiramate, and

(34) phytopharm compound 57;

and pharmaceutically acceptable salts and esters thereof;

provided that when the first appetite suppressant is a NPY1 antagonist, then the second appetite suppressant is not selected from the group consisting of: a MCH1R antagonist, a MCH2R antagonist, leptin, a leptin derivative, 5HT2c agonist, a Mc4r agonist, a serotonin reuptake inhibitor, and a GLP-1 agonist;

provided that when the first appetite suppressant is leptin, then the second appetite suppressant is not selected from the group consisting of: a MCH-1R antagonist, a MCH-2R antagonist, a NPY1 antagonist, a leptin derivative, 5HT2c agonist, a Mc4r agonist, a serotonin reuptake inhibitor, a GLP-1 agonist, a CCK-A agonist, an opioid antagonist, and a monoamine reuptake inhibitor; provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor;

provided that when the first appetite suppressant is an opioid antagonist, then the second appetite suppressant is not a serotonin reuptake inhibitor; and

provided that the appetite suppressants have different biological mechanisms of action.

In another subclass of this class, the appetite suppressant is selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,.

(7) a MCH2R agonist/antagonist,

(8) a NPY1 antagonist,

(9) a NPY2 agonist,

(10) a NPY4 agonist,

(11) a mGluR5 antagonist,

(12) an opioid antagonist,

(13) an orexin antagonist,

(14) a BRS3 agonist,

(15) a CCK-A agonist,

(16) CNTF,

(17) a CNTF agonist/modulator,

(18) a CNTF derivative,

(19) a 5HT2c agonist,

(20) a Mc4r agonist,

(21) a monoamine reuptake inhibitor,

(22) a serotonin reuptake inhibitor,

(23) a GLP-1 agonist,

(24) axokine,

(25) fenfluramine,

(26) nalmafene,

(27) phentermine,

(28) rimonabant,

(29) sibutramine, and

(30) topiramate;

and pharmaceutically acceptable salts and esters thereof;

provided that when the first appetite suppressant is a NPY1 antagonist, then the second appetite suppressant is not selected from the group consisting of: a MCH1R antagonist, a MCH2R antagonist, 5HT2c agonist, a Mc4r agonist, a serotonin reuptake inhibitor, and a GLP-1 agonist; provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor;

provided that when the first appetite suppressant is an opioid antagonist, then the second appetite suppressant is not a serotonin reuptake inhibitor; and

provided that the appetite suppressants have different biological mechanisms of action.

In another subclass of this class, the first appetite suppressant is a Mc4r agonist, and pharmaceutically acceptable salts and esters thereof, and the second appetite suppressant is selected from the group consisting of

(1) a MCH1R antagonist, and

(2) a MCH2R agonist/antagonist,

and pharmaceutically acceptable salts and esters thereof.

In another subclass of this class, the first appetite suppressant is a CB-1 antagonist/inverse agonist, and pharmaceutically acceptable salts and esters thereof, and the second appetite suppressant is selected from the group consisting of

(1) a NPY1 antagonist,

(2) a NPY2 agonist,

(3) a NPY4 agonist,

(4) a MCH1R antagonist,

(5) a MCH2R agonist/antagonist, and

(6) a Mc4r agonist;

and pharmaceutically acceptable salts and esters thereof.

In another subclass of this class, the first appetite suppressant is a CB-1 antagonist/inverse agonist, and pharmaceutically acceptable salts and esters thereof, and the second appetite suppressant is a Mc4r agonist, and pharmaceutically acceptable salts and esters thereof.

In yet another subclass of this class, the second appetite suppressant is selected from the group consisting of

(1) a 5HT transporter inhibitor,

(2) a NE transporter inhibitor,

(3) a ghrelin antagonist,

(4) a H3 antagonist/inverse agonist,

(5) a MCH1R antagonist,

(6) a MCH2R agonist/antagonist,

(7) an orexin antagonist,

(8) a BRS3 agonist,

(9) a CCK-A agonist,

(10) CNTF,

(11) a CNTF agonist/modulator,

(12) a CNTF derivative,

(13) a 5HT2c agonist,

(14) a Mc4r agonist,

(15) a monoamine reuptake inhibitor,

(16) a serotonin reuptake inhibitor, and

(17) a GLP-1 agonist;

and pharmaceutically acceptable salts and esters thereof;

provided that the appetite suppressant have different biological mechanisms of action.

In another embodiment of the present invention, the compositions comprise an appetite suppressant, and pharmaceutically acceptable salts and esters thereof, and a metabolic rate enhancer, and pharmaceutically acceptable salts and esters thereof.

In a class of this embodiment of this invention, the composition comprises

(a) an appetite suppressant selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY5 antagonist,

(10) a NPY2 agonist,

(11) a NPY4 agonist,

(12) a mGluR5 antagonist,

(13) leptin,

(14) leptin agonist/modulator,

(15) a leptin derivative,

(16) an opioid antagonist,

(17) an orexin antagonist,

(18) a BRS3 agonist,

(19) a CCK-A agonist,

(20) CNTF,

(21) a CNTF agonist/modulator,

(22) a CNTF derivative,

(23) 5HT2c agonist,

(24) a Mc4r agonist,

(25) a monoamine reuptake inhibitor,

(26) a serotonin reuptake inhibitor,

(27) a GLP-1 agonist,

(28) axokine,

(29) fenfluramine,

(30) nalmafene,

(31) phentermine,

(32) rimonabant,

(33) sibutramine,

(34) topiramate, and

(35) phytopharm compound 57;

and pharmaceutically acceptable salts and esters thereof, and

(b) a metabolic rate enhancer selected from the group consisting of

(1) an ACC2 inhibitor,

(2) a β3 agonist,

(3) a DGAT1 inhibitor,

(4) a DGAT2 inhibitor,

(5) a FAS inhibitor,

(6) a PDE inhibitor,

(7) a thyroid hormone β agonist,

(8) an UCP-1, 2, or 3 activator,

(9) an acyl-estrogen,

(10) a glucocorticoid antagonist,

(11) an 11β HSD-1 inhibitor,

(12) a Mc3r agonist,

(12) a SCD-1, and

(13) oleoyl-estrone;

and pharmaceutically acceptable salts and esters thereof;

provided that when the metabolic rate enhancer is a β3 agonist, then the appetite suppressant is not selected from the group consisting of a CB-1 antagonist/inverse agonist, a MCH1R antagonist, a MCH2R antagonist, a NPY5 antagonist, leptin, a leptin derivative, a CCK-A agonist, a 5HT2c agonist, a Mc4r agonist, a monoamine reuptake inhibitor, a serotonin reuptake inhibitor, and a GLP-1 agonist;

provided that when the metabolic rate enhancer is a UCP-1, 2 or 3 activator, then the appetite suppressant is not selected from the group consisting of leptin and a leptin derivative; provided that when the metabolic rate enhancer is an 11β HSD-1 inhibitor, then the appetite suppressant is not selected from the group consisting of: a CB-1 antagonist/inverse agonist, a NPY5 antagonist, a Mc4r agonist,

a monoamine reuptake inhibitor, and a serotonin reuptake inhibitor; and

provided that when the appetite suppressant is a monoamine reuptake inhibitor, then the metabolic rate enhancer is not a PDE inhibitor.

In a subclass of this class, the appetite suppressant is selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) leptin,

(13) a leptin derivative,

(14) an opioid antagonist,

(15) an orexin antagonist,

(16) a BRS3 agonist,

(17) a CCK-A agonist,

(18) CNTF,

(19) a CNTF agonist/modulator,

(20) a CNTF derivative,

(21) 5HT2c agonist,

(22) a Mc4r agonist,

(23) a monoamine reuptake inhibitor,

(24) a serotonin reuptake inhibitor,

(25) a GLP-1 agonist,

(26) axokine,

(27) fenfluramine,

(28) nalmafene,

(29) phentermine,

(30) rimonabant,

(31) sibutramine,

(32) topiramate, and

(33) phytopharm compound 57;

and pharmaceutically acceptable salts and esters thereof; and

(b) a metabolic rate enhancer selected from the group consisting of

(1) an ACC2 inhibitor,

(2) a β3 agonist,

(3) a DGAT1 inhibitor,

(4) a DGAT2 inhibitor,

(5) a FAS inhibitor,

(6) a PDE inhibitor,

(7) a thyroid hormone β agonist,

(8) an UCP-1, 2, or 3 activator,

(9) an acyl-estrogen,

(10) a glucocorticoid antagonist,

(11) an 11β HSD-1 inhibitor,

(12) a Mc3r agonist,

(13) a SCD-1, and

(14) oleoyl-estrone;

and pharmaceutically acceptable salts and esters thereof; and

provided that when the metabolic rate enhancer is a β3 agonist, then the appetite suppressant is not selected from the group consisting of a CB-1 antagonist/inverse agonist, a MCH1R antagonist, a MCH2R antagonist, leptin, a leptin derivative, a CCK-A agonist, a 5HT2c agonist, a Mc4r agonist, a monoamine reuptake inhibitor, a serotonin reuptake inhibitor, and a GLP-1 agonist;

provided that when the metabolic rate enhancer is a UCP-1, 2 or 3 activator, then the appetite suppressant is not selected from the group consisting of leptin and a leptin derivative;

provided that when the metabolic rate enhancer is an 11β HSD-1 inhibitor, then the appetite suppressant is not selected from the group consisting of: a CB-1 antagonist/inverse agonist, a Mc4r agonist, a monoamine reuptake inhibitor, and a serotonin reuptake inhibitor; and provided that when the appetite suppressant is a monoamine reuptake inhibitor, then the metabolic rate enhancer is not a PDE inhibitor.

In another subclass of this class, the composition comprises

(a) an appetite suppressant selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) an opioid antagonist,

(13) an orexin antagonist,

(14) a BRS3 agonist,

(15) a CCK-A agonist,

(16) CNTF,

(17) a CNTF agonist/modulator,

(18) a CNTF derivative,

(19) a 5HT2c agonist,

(20) a Mc4r agonist,

(21) a serotonin reuptake inhibitor, and

(22) a GLP-1 agonist;

and pharmaceutically acceptable salts and esters thereof; and

(b) a metabolic rate enhancer selected from the group consisting of

(1) an ACC2 inhibitor,

(2) a FAS inhibitor,

(3) a thyroid hormone β agonist,

(4) an UCP-1, 2, or 3 activator,

(5) an acyl-estrogen,

(6) a glucocorticoid antagonist, and

(7) oleoyl-estrone;

and pharmaceutically acceptable salts and esters thereof.

In yet another subclass of this class, the composition comprises an appetite suppressant selected from the group consisting of a NPY5 antagonist, and pharmaceutically acceptable salts and esters thereof, and a metabolic rate enhancer selected from the group consisting of an 11β HSD-1 inhibitor, and pharmaceutically acceptable salts and esters thereof.

In another embodiment of the present invention, the compositions comprise an appetite suppressant, and pharmaceutically acceptable salts and esters thereof, and a nutrient absorption inhibitor, and pharmaceutically acceptable salts and esters thereof.

In a class of this embodiment, the composition comprises

(a) an appetite suppressant selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY5 antagonist,

(10) a NPY2 agonist,

(11) a NPY4 agonist,

(12) a mGluR5 antagonist,

(13) leptin,

(14) a leptin agonist/modulator,

(15) a leptin derivative,

(16) an opioid antagonist,

(17) an orexin antagonist,

(18) a BRS3 agonist,

(19) a CCK-A agonist,

(20) CNTF,

(21) a CNTF agonist/modulator,

(22) a CNTF derivative,

(23) a 5HT2c agonist,

(24) a Mc4r agonist,

(25) a monoamine reuptake inhibitor,

(26) a serotonin reuptake inhibitor,

(27) a GLP-1 agonist,

(28) axokine,

(29) fenfluramine,

(30) nalmafene,

(31) phentermine,

(32) rimonabant,

(33) sibutramine,

(34) topiramate, and

(35) phytopharm compound 57;

and pharmaceutically acceptable salts and esters thereof, and

(b) a nutrient absorption inhibitor selected from the group consisting of

(1) a lipase inhibitor,

(2) a fatty acid transporter inhibitor,

(3) a dicarboxylate transporter inhibitor,

(4) a glucose transporter inhibitor,

(5) a phosphate transporter inhibitor, and

(6) orlistat;

and pharmaceutically acceptable salts and esters thereof;

provided that when the appetite suppressant is a monoamine reuptake inhibitor, then the nutrient absorption inhibitor is not a lipase inhibitor.

In a subclass of this class, the composition comprises

(a) an appetite suppressant selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) leptin,

(13) a leptin agonist/modulator,

(14) a leptin derivative,

(15) an opioid antagonist,

(16) an orexin antagonist,

(17) a BRS3 agonist,

(18) a CCK-A agonist,

(19) CNTF,

(20) a CNTF agonist/modulator,

(21) a CNTF derivative,

(22) a 5HT2c agonist,

(23) a Mc4r agonist,

(24) a serotonin reuptake inhibitor,

(25) a GLP-1 agonist,

(26) axokine,

(27) fenfluramine,

(28) nalmafene,

(29) phentermine,

(30) rimonabant,

(31) sibutramine,

(32) topiramate, and

(33) phytopharm compound 57;

and pharmaceutically acceptable salts and esters thereof; and

(b) a nutrient absorption inhibitor selected from the group consisting of

(1) a lipase inhibitor,

(2) a fatty acid transporter inhibitor,

(3) a dicarboxylate transporter inhibitor,

(4) a glucose transporter inhibitor,

(5) a phosphate transporter inhibitor, and

(6) orlistat;

and pharmaceutically acceptable salts and esters thereof.

In another subclass of this class, the composition comprises

(a) an appetite suppressant selected from the group consisting of

(1) a 5HT transporter inhibitor,

(2) a NE transporter inhibitor,

(3) a CB-1 antagonist/inverse agonist,

(4) a ghrelin antagonist,

(5) a H3 antagonist/inverse agonist,

(6) a MCH1R antagonist,

(7) a MCH2R agonist/antagonist,

(8) a NPY1 antagonist,

(9) a NPY2 agonist,

(10) a NPY4 agonist,

(11) a mGluR5 antagonist,

(12) an opioid antagonist,

(13) an orexin antagonist,

(14) a BRS3 agonist,

(15) a CCK-A agonist,

(16) CNTF,

(17) a CNTF agonist/modulator,

(18) a CNTF derivative,

(19) a 5HT2c agonist,

(20) a Mc4r agonist,

(21) a serotonin reuptake inhibitor,

(22) a GLP-1 agonist;

and pharmaceutically acceptable salts and esters thereof; and

(b) a nutrient absorption inhibitor selected from the group consisting of

(1) a lipase inhibitor,

(2) a fatty acid transporter inhibitor,

(3) a dicarboxylate transporter inhibitor,

(4) a glucose transporter inhibitor, and

(5) orlistat;

and pharmaceutically acceptable salts and esters thereof.

In another embodiment of the present invention, the composition comprises two metabolic rate enhancers, and pharmaceutically acceptable salts and esters thereof, provided that the metabolic rate enhancers have different biological mechanisms of action.

In a class of this embodiment, the composition comprises two metabolic rate enhancers, wherein each metabolic rate enhancer is selected from the group consisting of

(1) an ACC2 inhibitor,

(2) a β3 agonist,

(3) a FAS inhibitor,

(4) a PDE inhibitor,

(5) a thyroid hormone β agonist,

(6) an UCP-1, 2, or 3 activator,

(7) an acyl-estrogen,

(8) a glucocorticoid antagonist,

(9) an 11βHSD-1 inhibitor,

(10) a Mc3r agonist,

(11) a SCD-1, and

(12) oleoyl-estrone;

and pharmaceutically acceptable salts and esters thereof,

provided that the metabolic rate enhancers have different biological mechanisms of action.

In another embodiment of the present invention, the composition comprises a metabolic rate enhancer, and pharmaceutically acceptable salts and esters thereof, and a nutrient absorption inhibitor, and pharmaceutically acceptable salts and esters thereof.

In a class of this embodiment, the composition comprises:

(a) a metabolic rate enhancer selected from the group consisting of

(1) an ACC2 inhibitor,

(2) a β3 agonist,

(3) a FAS inhibitor,

(4) a PDE inhibitor,

(5) a thyroid hormone β agonist,

(6) an UCP-1, 2, or 3 activator,

(7) an acyl-estrogen,

(8) a glucocorticoid antagonist,

(9) an 11β HSD-1 inhibitor,

(10) a Mc3r agonist,

(11) a SCD-1, and

(12) oleoyl-estrone;

and pharmaceutically acceptable salts and esters thereof; and

(b) a nutrient absorption inhibitor selected from the group consisting of

(1) a lipase inhibitor,

(2) a fatty acid transporter inhibitor,

(3) a dicarboxylate transporter inhibitor,

(4) a glucose transporter inhibitor,

(5) a phosphate transporter inhibitor, and

(6) orlistat;

and pharmaceutically acceptable salts and esters thereof.

In a subclass of this class, the composition comprises:

(a) a metabolic rate enhancer selected from the group consisting of

(1) an ACC2 inhibitor,

(2) a β3 agonist,

(3) a FAS inhibitor,

(4) a PDE inhibitor,

(5) a thyroid hormone β agonist,

(6) an UCP-1, 2, or 3 activator,

(7) an acyl-estrogen,

(8) a glucocorticoid antagonist,

(9) an 11β HSD-1 inhibitor, and

(10) oleoyl-estrone;

and pharmaceutically acceptable salts and esters thereof; and

(b) a nutrient absorption inhibitor selected from the group consisting of

(1) a lipase inhibitor,

(2) a fatty acid transporter inhibitor,

(3) a dicarboxylate transporter inhibitor,

(4) a glucose transporter inhibitor and

(5) orlistat;

and pharmaceutically acceptable salts and esters thereof.

In another embodiment of the present invention, the composition comprises two nutrient absorption inhibitors, and pharmaceutically acceptable salts and esters thereof, provided that the nutrient absorption inhibitors have different biological mechanisms of action.

In a class of this embodiment, the composition comprises two nutrient absorption inhibitors, wherein each nutrient absorption inhibitor is selected from the group consisting of

(1) a lipase inhibitor,

(2) a fatty acid transporter inhibitor,

(3) a dicarboxylate transporter inhibitor,

(4) a glucose transporter inhibitor,

(5) a phosphate transporter inhibitor and

(6) orlistat;

and pharmaceutically acceptable salts and esters thereof; provided that the nutrient absorption inhibitors have different biological mechanisms of action.

In a subclass of this class, the composition comprises two nutrient absorption inhibitors, wherein each nutrient absorption inhibitors is selected from the group consisting of

(1) a lipase inhibitor,

(2) a fatty acid transporter inhibitor,

(3) a dicarboxylate transporter inhibitor,

(4) a glucose transporter inhibitor, and

(5) orlistat;

and pharmaceutically acceptable salts and esters thereof;

provided that the nutrient absorption inhibitors have different biological mechanisms of action.

The present invention further relates to methods of treating or preventing obesity in a subject in need thereof by administering an effective amount of an appetite suppressant and/or a metabolic rate enhancer and/or a nutrient absorption inhibitor.

The present invention relates to a method of treating a subject having a disorder associated with excessive food intake comprising administration of

(a) a therapeutically effective amount of an appetite suppressant, and pharmaceutically acceptable salts and esters thereof; and

(b) a therapeutically effective amount of a metabolic rate enhancer, and pharmaceutically acceptable salts and esters thereof; to a subject in need of such treatment.

In one embodiment of the present invention, the method of treating a subject having a disorder associated with excessive food intake comprising administration of

(a) a therapeutically effective amount of an appetite suppressant selected from the group consisting of a 5HT transporter inhibitor, a NE transporter inhibitor, a CB-1 antagonist/inverse agonist, a ghrelin antagonist, a H3 antagonist/inverse agonist, a MCH1R antagonist, a MCH2R agonist/antagonist, a NPY1 antagonist, a NPY2 agonist, NPY4 agonist, a mGluR5 antagonist, leptin, a leptin derivative, an opioid antagonist, an orexin antagonist, a BRS3 agonist, a CCK-A agonist, CNTF, a CNTF derivative, a 5HT2c agonist, a Mc4r agonist, a monoamine reuptake inhibitor, a serotonin reuptake inhibitor, a GLP-1 agonist, topiramate, and phytopharm compound 57; and pharmaceutically acceptable salts and esters thereof; and

(b) a therapeutically effective amount of a metabolic rate enhancer selected from the group consisting of an ACC2 inhibitor, a β3 agonist, a DGAT1 inhibitor, a DGAT2 inhibitor, a FAS inhibitor, a PDE inhibitor, a thyroid hormone β agonist, an UCP-1, 2, or 3 activator, an acyl-estrogen, a glucocorticoid antagonist, and an 11β HSD-1 inhibitor, a Mc3r agonist, and a SCD-1; and pharmaceutically acceptable salts and esters thereof; to a subject in need of such treatment; and pharmaceutically acceptable salts and esters thereof; provided that when the metabolic rate enhancer is a β3 agonist, then the appetite suppressant is not selected from the group consisting of a CB-1 antagonist/inverse agonist, a MCH1R antagonist, a MCH2R antagonist, leptin, a leptin derivative, a CCK-A agonist, a 5HT2c agonist, a Mc4r agonist, a monoamine reuptake inhibitor, a serotonin reuptake inhibitor, and a GLP-1 agonist; provided that when the metabolic rate enhancer is a UCP-1, 2 or 3 activator, then the appetite suppressant is not selected from the group consisting of leptin and a leptin derivative; provided that when the metabolic rate enhancer is an 11β HSD-1 inhibitor, then the appetite suppressant is not selected from the group consisting of: a CB-1 antagonist/inverse agonist, a Mc4r agonist, a monoamine reuptake inhibitor, and a serotonin reuptake inhibitor; and provided that when the appetite suppressant is a monoamine reuptake inhibitor, then the metabolic rate enhancer is not a PDE inhibitor.

The present invention relates to a method of treating a subject having a disorder associated with excessive food intake comprising administration of

(a) a therapeutically effective amount of an appetite suppressant, and pharmaceutically acceptable salts and esters thereof; and

(b) a therapeutically effective amount of a nutrient absorption inhibitor, and pharmaceutically acceptable salts and esters thereof; to a subject in need of such treatment.

In one embodiment of the present invention, the method of treating a subject having a disorder associated with excessive food intake comprises administration of

(a) a therapeutically effective amount of an appetite suppressant selected from the group consisting of a 5HT transporter inhibitor, a NE transporter inhibitor, a CB-1 antagonist/inverse agonist, a ghrelin antagonist, a H3 antagonist/inverse agonist, a MCH1R antagonist, a MCH2R agonist/antagonist, a NPY1 antagonist, NPY2 agonist, NPY4 agonist, a mGluR5 antagonist, leptin, a leptin derivative, an opioid antagonist, an orexin antagonist, a BRS3 agonist, a CCK-A agonist, CNTF, a CNTF derivative, a 5HT2c agonist, a Mc4r agonist, a monoamine reuptake inhibitor, a serotonin reuptake inhibitor, a GLP-1 agonist, topiramate, and phytopharm compound 57; and pharmaceutically acceptable salts and esters thereof; and

(b) a therapeutically effective amount of a nutrient absorption inhibitor selected from the group consisting of a lipase inhibitor, a fatty acid transporter inhibitor, a dicarboxylate transporter inhibitor, a glucose transporter inhibitor, and a phosphate transporter inhibitor; and pharmaceutically acceptable salts and esters thereof; to a subject in need of such treatment; provided that when the appetite suppressant is a monoamine reuptake inhibitor, then the nutrient absorption inhibitor is not a lipase inhibitor.

The present invention relates to a method of treating a subject having a disorder associated with excessive food intake comprising administration of

(a) a therapeutically effective amount of a metabolic rate enhancer, and pharmaceutically acceptable salts and esters thereof; and

(b) a therapeutically effective amount of a nutrient absorption inhibitor, and pharmaceutically acceptable salts and esters thereof; to a subject in need of such treatment.

In one embodiment of the present invention, the method of treating a subject having a disorder associated with excessive food intake comprises administration of

(a) a therapeutically effective amount of a metabolic rate enhancer selected from the group consisting of an ACC2 inhibitor, a β3 agonist, a DGAT1 inhibitor, a DGAT2 inhibitor, a FAS inhibitor, a PDE inhibitor, a thyroid hormone β agonist, an UCP-1, 2, or 3 activator, an acyl-estrogen, a glucocorticoid antagonist, an 11β HSD-1 inhibitor, a Mc3r agonist, a SCD-1; and pharmaceutically acceptable salts and esters thereof; and

(b) a therapeutically effective amount of a nutrient absorption inhibitor selected from the group consisting of a lipase inhibitor, a fatty acid transporter inhibitor, a dicarboxylate transporter inhibitor, a glucose transporter inhibitor, and a phosphate transporter inhibitor; and pharmaceutically acceptable salts and esters thereof; to a subject in need of such treatment.

The present invention also relates to a method of treating a subject having a disorder associated with excessive food intake comprising administration of a therapeutically effective amount of two appetite suppressants, and pharmaceutically acceptable salts and esters thereof; to a subject in need of such treatment.

In one embodiment of the present invention, the method of treating a subject having a disorder associated with excessive food intake comprises administration of a therapeutically effective amount of two appetite suppressants selected from the group consisting of: a 5HT transporter inhibitor, a NE transporter inhibitor, a CB-1 antagonist/inverse agonist, a ghrelin antagonist, a H3 antagonist/inverse agonist, a MCH1R antagonist, a MCH2R agonist/antagonist, a NPY1 antagonist, NPY2 agonist, a NPY4 agonist, a mGluR5 antagonist, a leptin, a leptin derivative, an opioid antagonist, an orexin antagonist, a BRS3 agonist, a CCK-A agonist, CNTF, a CNTF derivative, a 5HT2c agonist, a Mc4r agonist, a monoamine reuptake inhibitor, a serotonin reuptake inhibitor, a GLP-1 agonist,topiramate, and phytopharm compound 57; and pharmaceutically acceptable salts and esters thereof;

to a subject in need of such treatment; provided that when the first appetite suppressant is a NPY1 antagonist, then the second appetite suppressant is not selected from the group consisting of: a MCH1R antagonist, a MCH2R antagonist, leptin, a leptin derivative, 5HT2c agonist, a Mc4r agonist, a serotonin reuptake inhibitor, and a GLP-1 agonist; provided that when the first appetite suppressant is leptin, then the second appetite suppressant is not selected from the group consisting of: a MCH-1R antagonist, a MCH-2R antagonist, a NPY1 antagonist, a leptin derivative, 5HT2c agonist, a Mc4r agonist, a serotonin reuptake inhibitor, a GLP-1 agonist, a CCK-A agonist, an opioid antagonist, and a monoamine reuptake inhibitor; provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor; and provided that the appetite suppressants have different biological mechanisms of action.

The present invention also relates to a method of treating a subject having a disorder associated with excessive food intake comprising administration of a therapeutically effective amount of two metabolic rate enhancers, and pharmaceutically acceptable salts and esters thereof; to a subject in need of such treatment.

In one embodiment of the present invention, the method of treating a subject having a disorder associated with excessive food intake comprises administration of a therapeutically effective amount of two metabolic rate enhancers selected from the group consisting of an ACC2 inhibitor, a β3 agonist, a DGAT1 inhibitor, a DGAT2 inhibitor, a FAS inhibitor, a PDE inhibitor, a thyroid hormone β agonist, an UCP-1, 2, or 3 activator, an acyl-estrogen, a glucocorticoid antagonist, an 11β HSD-1 inhibitor, a Mc3r agonist, and a SCD-1; and pharmaceutically acceptable salts and esters thereof; to a subject in need of such treatment; provided that when the first metabolic rate enhancer is an 11β HSD-1 inhibitor, then the second metabolic rate enhancer is not a β3 agonist; and provided that the metabolic rate enhancers have different biological mechanisms of action.

The present invention also relates to a method of treating a subject having a disorder associated with excessive food intake comprising administration of a therapeutically effective amount of two nutrient absorption inhibitors, and pharmaceutically acceptable salts and esters thereof; to a subject in need of such treatment.

In one embodiment of the present invention, the method of treating a subject having a disorder associated with excessive food intake comprises administration of a therapeutically effective amount of two nutrient absorption inhibitors selected from the group consisting of a lipase inhibitor, a fatty acid transporter inhibitor, a dicarboxylate transporter inhibitor, a glucose transporter inhibitor, and a phosphate transporter inhibitor; and pharmaceutically acceptable salts and esters thereof; to a subject in need of such treatment; provided that the nutrient absorption inhibitors have different biological mechanisms of action.

The present invention relates to a method of preventing obesity in a subject at risk for obesity comprising administration to said subject

(a) a prophylactically effective amount of an appetite suppressant, and pharmaceutically acceptable salts and esters thereof,

(b) a prophylactically effective amount of a metabolic rate enhancer, and pharmaceutically acceptable salts and esters thereof.

The present invention relates to a method of preventing obesity in a subject at risk for obesity comprising administration to said subject

(a) a prophylactically effective amount of an appetite suppressant, and pharmaceutically acceptable salts and esters thereof,

(b) a prophylactically effective amount of a nutrient absorption inhibitor, and pharmaceutically acceptable salts and esters thereof.

The present invention relates to a method of preventing obesity in a subject at risk for obesity comprising administration to said subject

(a) a prophylactically effective amount of a metabolic rate enhancer, and pharmaceutically acceptable salts and esters thereof,

(b) a prophylactically effective amount of a nutrient absorption inhibitor, and pharmaceutically acceptable salts and esters thereof.

The present invention relates to a method of preventing obesity in a subject at risk for obesity comprising administration to said subject a prophylactically effective amount of two appetite suppressants, and pharmaceutically acceptable salts and esters thereof. The present invention relates to a method of preventing obesity in a subject at risk for obesity comprising administration to said subject a prophylactically effective amount of two metabolic rate enhancers, and pharmaceutically acceptable salts and esters thereof. The present invention relates to a method of preventing obesity in a subject at risk for obesity comprising administration to said subject a prophylactically effective amount of two nutrient absorption inhibitors, and pharmaceutically acceptable salts and esters thereof.

The present invention also relates to pharmaceutical compositions, and medicaments useful for carrying out these methods.

The present invention relates to the use of an appetite suppressant, and pharmaceutically acceptable salts and esters thereof; and a metabolic rate enhancer, and pharmaceutically acceptable salts and esters thereof; for the manufacture of a medicament for treatment of obesity which comprises an effective amount of an appetite suppressant and an effective amount of a metabolic rate enhancer, together or separately.

The present invention relates to the use of an appetite suppressant, and pharmaceutically acceptable salts and esters thereof; and a nutrient absorption inhibitor, and pharmaceutically acceptable salts and esters thereof; for the manufacture of a medicament for treatment of obesity which comprises an effective amount of an appetite suppressant and an effective amount of a nutrient absorption inhibitor, together or separately.

The present invention relates to the use of a metabolic rate enhancer, and pharmaceutically acceptable salts and esters thereof; and a nutrient absorption inhibitor, and pharmaceutically acceptable salts and esters thereof; for the manufacture of a medicament for treatment of obesity which comprises an effective amount of a metabolic rate enhancer and an effective amount of a nutrient absorption inhibitor, together or separately.

The present invention relates to the use of two appetite suppressants, and pharmaceutically acceptable salts and esters thereof; for the manufacture of a medicament for treatment of obesity which comprises an effective amount of the appetite suppressants, together or separately. The present invention also relates to the use of two metabolic rate enhancers, and pharmaceutically acceptable salts and esters thereof; for the manufacture of a medicament for treatment of obesity which comprises an effective amount of the metabolic rate enhancers, together or separately. The present invention also relates to the use of two nutrient absorption inhibitors, and pharmaceutically acceptable salts and esters thereof; for the manufacture of a medicament for treatment of obesity which comprises an effective amount of the nutrient absorption inhibitors, together or separately.

The present invention further relates to a product containing an appetite suppressant, and pharmaceutically acceptable salts and esters thereof; and a metabolic rate enhancer, and pharmaceutically acceptable salts and esters thereof; as a combined preparation for simultaneous, separate or sequential use in obesity. The present invention further relates to a product containing an appetite suppressant, and pharmaceutically acceptable salts and esters thereof; and a nutrient absorption inhibitor and pharmaceutically acceptable salts and esters thereof; as a combined preparation for simultaneous, separate or sequential use in obesity. The present invention further relates to a product containing a metabolic rate enhancer, and pharmaceutically acceptable salts and esters thereof; and a nutrient absorption inhibitor, and pharmaceutically acceptable salts and esters thereof; as a combined preparation for simultaneous, separate or sequential use in obesity.

The present invention further relates to a product containing two appetite suppressants, and pharmaceutically acceptable salts and esters thereof, with different biological mechanisms of action as a combined preparation for simultaneous, separate or sequential use in obesity, and obesity related disorders. The present invention further relates to a product containing two metabolic rate enhancers, and pharmaceutically acceptable salts and esters thereof, with different biological mechanisms of action as a combined preparation for simultaneous, separate or sequential use in obesity, and obesity related disorders. The present invention further relates to a product containing two nutrient absorption inhibitors, and pharmaceutically acceptable salts and esters thereof, with different biological mechanisms of action as a combined preparation for simultaneous, separate or sequential use in obesity, and obesity related disorders.

The invention further provides pharmaceutical compositions comprising an appetite suppressant and a metabolic rate enhancer, as active ingredients. The invention further provides pharmaceutical compositions comprising an appetite suppressant and a nutrient absorption inhibitor, as active ingredients. The invention further provides pharmaceutical compositions comprising a metabolic rate enhancer and a nutrient absorption inhibitor, as active ingredients.

The invention further provides pharmaceutical compositions comprising two appetite suppressants with different biological mechanisms of action, as active ingredients. The invention further provides pharmaceutical compositions comprising two metabolic rate enhancers with different biological mechanisms of action, as active ingredients. The invention further provides pharmaceutical compositions comprising two nutrient absorption inhibitors with different biological mechanisms of action, as active ingredients.

As used herein, the term “appetite suppressant” includes compounds that reduce total food intake by more than 5%, or reduce caloric intake or selectively reduce intake of specific components of the diet such as carbohydrates or fats by more than 5%.

As used herein, the term “metabolic rate enhancer” includes compounds which, when administered to a subject, act to increase the metabolic rate of the subject, particularly those agents which increase metabolic rate by at least 5%, preferably 10%, most preferably 20% in 24 hour energy expenditure, and/or increase the oxidation of fatty acids relative to carbohydrates when administered to the subject.

As used herein, the term “nutrient absorption inhibitor” includes compounds that inhibit the absorption of more than 10% of the nutrients.

One of ordinary skill in the art can readily identify the agents useful in the compositions and methods of the present invention. Appetite suppressants can be evaluated in rodents according to the procedures described in: Daniels, A. J. et al., Regulatory Peptides, 106:47-54 (2002); Halaas, J. L. et. al., Science, 269: 543-546 (1995); and Strack, A. M., Obesity Research, 10:173-81 (2002). Metabolic rate enhancers are routinely evaluated in rodents (Atgie, C., Comp. Biochem. Physiol. A. Mol. Integr. Physiol. 119:629-36 (1998); Himms-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of metabolic rate enhancers is supported by experiments with mice, in which the RII-beta gene has been deleted, that were shown to be resistant to diet induced obesity (D. E. Cummings et al. Nature 382: 622-626 (1996)). Nutrient absorption inhibitors can be evaluated in: Badr M. Z. and Chen, T. S., Toxicology, 34:333-40 (1985); Sorribas, V., J. Pharm. Pharmacol., 44:1030-2 (1992).

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonist/inverse agonists useful in the present invention include: U.S. Pat. Nos. 5,532,237, 4,973,587, 5,013,837, 5,081,122, 5,112,820, 5,292,736, 5,624,941 and U.S. Pat. No. 6,028,084; and PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, and WO 02/076949; and EPO Application No. EP-658546. Specific CB-1 antagonists/inverse agonists useful in the present invention include, but are not limited to, rimonabant (Sanofi Synthelabo), and SR-147778 (Sanofi Synthelabo).

Ghrelin antagonists useful in the present invention, include: PCT Application Nos. WO 01/87335, and WO 02/08250. Ghrelin antagonists are also known as GHS (growth hormone secretagogue receptor) antagonists. The compositions and methods of the present invention therefore comprehend the use GHS antagonists in place of ghrelin antagonists.

Histamine 3 (H3) antagonist/inverse agonists useful in the present invention include: PCT Application No. WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem.. 43:3335-43 (2000)). Specific H3 antagonists/inverse agonists useful in the present invention include, but are not limited to, thioperamide, 3-(1H-imidazol-4-yl)propyl N-(4-pentenyl)carbamate, clobenpropit, iodophenpropit, imoproxifan, and GT2394 (Gliatech).

Melanin-concentrating hormone 1 receptor (MCH1R) antagonists and melanin-concentrating hormone 2 receptor (MCH2R) agonist/antagonists useful in the present invention include PCT Patent Application Nos. WO 01/82925, WO 01/87834, WO 02/06245, WO 02/04433, and WO 02/51809; and Japanese Patent Application No. JP 13226269. Specific MCH1R antagonists useful in the present invention include, but are not limited to, T-226296 (Takeda).

Neuropeptide Y1 (NPY1) antagonists useful in the present invention, include: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BIBP3226, J-115814, BIBO 3304, LY-357897, CP-671906, and GI-264879A.

Neuropeptide Y5 (NPY5) antagonists useful in the present invention, include, but are not limited to, the compounds described in: U.S. Pat. Nos. 6,140,354, 6,191,160, 6,258,837, 6,313,298, 6,337,332, 6,329,395, and 6,340,683; U.S. Pat. Nos. 6,326,375; 6,335,345; European Patent Nos. EP-01010691, and EP-01044970; and PCT International Patent Publication Nos. WO 97/19682, WO 97/20820, WO 97/20821, WO 97/20822, WO 97/20823, WO 98/27063, WO 00/64880, WO 00/68197, WO 00/69849, WO 01/09120, WO 01/85714, WO 01/85730, WO 01/07409, WO 01/02379, WO 01/02379, WO 01/23388, WO,01/23389, WO 01/44201, WO 01/62737, WO 01/62738, WO 01/09120, WO 02/22592, WO 0248152, WO 02/49648, and WO 01/14376. Specific NPY 5 antagonists useful in the combinations of the present invention, include, but are not limited to 152,804, GW-569180A, GW-594884A, GW-587081X, GW-548118X; FR226928, FR 240662, FR252384; 1229U91, GI-264879A, CGP71683A, LY-377897, PD-160170, SR-120562A, SR-120819A and JCF-104. Additional specific NPY 5 antagonists useful in the combinations of the present invention, include, but are not limited to the compounds described in Norman et al., J. Med. Chem. 43:4288-4312 (2000).

Neuropeptide Y2 (NPY2) agonists useful in the present invention, include, but are not limited to compounds such as PYY3-36 as described in Batterham, et al., Nature. 418:650-654 (2003), NPY3-36, and other Y2 agonists such as N acetyl [Leu(28,31)] NPY 24-36 (White-Smith and Potter, Neuropeptides 33:526-33 (1999)), TASP-V (Malis et al., Br. J. Pharmacol. 126:989-96 (1999)), cyclo-(28/32)-Ac-[Lys28-Glu32]-(25-36)-pNPY (Cabrele and Beck-Sickinger J-Pept-Sci. 6:97-122 (2000)).

Neuropeptide Y4 (NPY4) agonists useful in the present invention, include, but are not limited to compounds such as pancreatic peptide (PP) as described in Batterham et al., J. Clin. Endocrinol. Metab. 88:3989-3992 (2003), and other Y4 agonists such as 1229U91 (Raposinho et al., Neuroendocrinology. 71:2-7(2000)).

Metabotropic glutamate subtype 5 receptor (mGluR5) antagonists useful in the present invention, include, but are not limited to compounds such as 2-methyl-6-(phenylethynyl)-pyridine (MPEP) and (3-[(2-methyl-1,3-thiazol-4-yl)ethynyl]pyridine) (MTEP) and those compounds described in Anderson J. et al., J, Eur J Pharmacol. Jul. 18, 2003;473(1):35-40; Cosford N. et al., Bioorg Med Chem Lett. Feb. 10, 2003;13(3):351-4; and Anderson J. et al., J Pharmacol Exp Ther. December 2002:303(3):1044-51.

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include: U.S. Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include: PCT Application No. WO 00/21509. Specific opioid antagonists useful in the present invention include, but are not limited to, nalmefene (Revex®), 3-methoxynaltrexone naloxone, and naltrexone.

Orexin antagonists useful in the present invention include: PCT Patent Application Nos. WO 01/96302, WO 01/68609, WO 02/51232, and WO 02/51838. Specific orexin antagonists useful in the present invention include, but are not limited to, SB-334867-A.

Phytopharm compound 57 is also known as CP 644,673.

Acyl-estrogens useful in the present invention include oleoyl-estrone (del Mar-Grasa, M. et al., Obesity Research, 9:202-9 (2001)).

Cholecystokinin-A (CCK-A) agonists useful in the present invention include U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 (Pfizer). CNTF derivatives useful in the present invention include, but are not limited to, axokine (Regeneron); and PCT Application Nos. WO 94/09134, WO 98/22128, and WO 99/43813.

5HT2C agonists useful in the present invention include: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5HT2C agonists useful in this invention include, but are not limited to, BVT933, DPCA37215, WAY161503, and R-1065.

Melanocortin receptor agonists useful in the present invention include Melanotan-II (MT-II) of the formula:

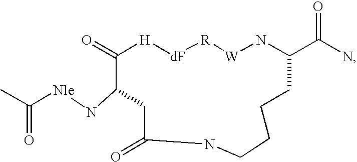

and pharmaceutically acceptable salts, esters and tautomers thereof. Melanotan-II is disclosed in U.S. Pat. No. 5,674,839, which is hereby incorporated by reference in its entirety. Mc4r agonists useful in the present invention include: PCT Application Nos. WO 01/991752, WO 01/74844, WO 02/12166, WO 02/11715, and WO 02/12178. Specific Mc4r agonists useful in the present invention include CHIR86036 (Chiron); ME-10142, and ME-10145 (Melacure).

Monoamine reuptake inhibitors useful in the present invention include: PCT Application Nos. WO 01/27068, and WO 01/62341. Specific monoamine reuptake inhibitors useful in the present invention include, but are not limited to, sibutramine (Meridia®/Reductil®) disclosed in U.S. Pat. Nos. 4,746,680, 4,806,570, and 5,436,272, and U.S. patent Publication Ser. No. 2002/0,006,964. The present invention encompasses sibutratmine as a racemic mixture, as optically pure isomers (+) and (−), or a pharmaceutically acceptable salt, solvent, hydrate, clathrate or prodrug thereof; particularly sibutramine hydrochloride monohydrate.

Serotonin reuptake inhibitors, and releasers, useful in the present invention include: dexfenfluramine, fluoxetine, and other serotonin reuptake inhibitors, including, but not limited to, those in U.S. patent application Ser. No. 6,365,633; and PCT Patent Application Nos. WO 01/27060, and WO 01/162341.

11β HSD-1 inhibitor useful in the present invention include, but are not limited to, those in WO 01/90091, WO 01/90090, and WO 01/90092.

Uncoupling Protein (UCP-1, UCP-2, and UCP-3) activators useful in the present invention include: PCT Patent Application No. WO 99/00123. Specific uncoupling protein (UCP-1, UCP-2, and UCP-3) activators useful in the present invention include, but are not limited to, phytanic acid, 4-[(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include: U.S. patent application Ser. No. 5,705,515, and U.S. Pat. No. 5,451,677; and PCT Patent Application Nos. WO 01/74782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A.

Thyroid hormone β agonists useful in the present invention include: PCT Application No. WO 02/15845; and Japanese Patent Application No. JP 2000256190. Specific thyroid hormone β agonists useful in the present invention include, but are not limited to, KB-2611 (KaroBioBMS).

Specific fatty acid synthase (FAS) inhibitors useful in the present invention, include, but are not limited to, Cerulenin and C75.

Specific phosphodieterase (PDE) inhibitors useful in the present invention, include, but are not limited to, theophylline, pentoxifylline, zaprinast, sildenafil, amrinone, milrinone, cilostamide, rolipram, and cilomilast.

Lipase inhibitors useful in the present invention include: PCT Application No. WO 01/77094. Specific lipase inhibitors useful in the present invention include, but are not limited to, orlistat (Xenical®), Triton WR1339, RHC80267, lipstatin, tetrahydrolipstatin, teasaponin, and diethylumbelliferyl phosphate.

Topiramate (Topimax®), indicated as an anti-convulsant and an anti-convulsant, has been shown to increase weight loss.

The above compounds are only illustrative of the appetite suppressants, metabolic rate enhancers and nutrient absorption inhibitors that can be used in the compositions of the present invention. As this listing of compounds is not meant to be comprehensive, the methods of the present invention may employ any appetite suppressant, any metabolic rate enhancer, and any nutrient absorption inhibitor, and are not limited to any particular structural class of compounds.

The present invention further relates to the treatment of obesity with a combination of an appetite suppressant and a metabolic rate enhancer, which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the appetite suppressant, or a pharmaceutically acceptable salt or ester thereof, and a pharmaceutically acceptable carrier or diluent in a first unit dosage form, and a second unit dosage form comprising a prophylactically or therapeutically effective amount of the metabolic rate enhancer, or a pharmaceutically acceptable salt or ester thereof, and a pharmaceutically acceptable carrier or diluent in a second unit dosage form.

The present invention further relates to the treatment of obesity with a combination of an appetite suppressant and a nutrient absorption inhibitor which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the appetite suppressant, or a pharmaceutically acceptable salt or ester thereof, and a pharmaceutically acceptable carrier or diluent in a first unit dosage form, and a second unit dosage form comprising a prophylactically or therapeutically effective amount of the nutrient absorption inhibitor, or a pharmaceutically acceptable salt or ester thereof, and a pharmaceutically acceptable carrier or diluent in a second unit dosage form.

The present invention further relates to the treatment of obesity with a combination of a metabolic rate enhancer and a nutrient absorption inhibitor which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the metabolic rate enhancer, or a pharmaceutically acceptable salt or ester thereof, and a pharmaceutically acceptable carrier or diluent in a first unit dosage form, and a second unit dosage form comprising a prophylactically or therapeutically effective amount of the nutrient absorption inhibitor, or a pharmaceutically acceptable salt or ester thereof, and a pharmaceutically acceptable carrier or diluent in a second unit dosage form.

The present invention also relates to a kit comprising at least one unit dosage of a prophylactically or therapeutically effective amount of a first appetite suppressant, and pharmaceutically acceptable salts and esters thereof, and at least one unit dosage of a prophylactically or therapeutically effective amount of a second appetite suppressant, and pharmaceutically acceptable salts and esters thereof. The present invention also relates to a kit comprising at least one unit dosage of a prophylactically or therapeutically effective amount of a first metabolic rate enhancer, and pharmaceutically acceptable salts and esters thereof, and at least one unit dosage of a prophylactically or therapeutically effective amount of a second metabolic rate enhancer, and pharmaceutically acceptable salts and esters thereof. The present invention also relates to a kit comprising at least one unit dosage of a prophylactically or therapeutically effective amount of a first nutrient absorption inhibitor, and pharmaceutically acceptable salts and esters thereof, and at least one unit dosage of a prophylactically or therapeutically effective amount of a second nutrient absorption inhibitor, and pharmaceutically acceptable salts and esters thereof.

The kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered. The term “pharmaceutically acceptable salts” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to appetite suppressants, to metabolic rate enhancers, and to nutrient absorption inhibitors are meant to also include the pharmaceutically acceptable salts and esters thereof.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The “pharmaceutically acceptable esters” in the present invention refer to non-toxic esters, for example, the pharmaceutically acceptable, common esters on carboxyl group when the compound has a carboxyl group, for example, esters with lower alkyls (for example methyl, ethyl, propyl, isopropyl, butyl, sec-butyl, tert-butyl, pentyl, isopentyl, neopentyl, cyclopropyl, cyclobutyl, cyclopentyl), aralkyls (for example benzyl, phenethyl), lower alkenyls (for example allyl, 2-butenyl), lower alkoxy (lower) alkyls (for example methoxymethyl, 2-methoxyethyl, 2-ethoxyethyl), lower alkanoyloxy (lower) alkyls (for example acetoxymethyl, pivaloyloxy-methyl, 1-pivaloyloxyethyl), lower alkoxycarbonyl (lower) alkyls (for example methoxycarbonylmethyl, isopropoxycarbonylmethyl), carboxy-(lower)alkyls (for example carboxymethyl), lower alkoxycarbonyloxy-(lower)alkyls (for example 1-(ethoxycarbonyloxy)ethyl, 1-(cyclohexyl-oxycarbonyloxy)ethyl), carbamoyloxy(lower)alkyls (for example carbamoyloxymethyl), phthalidyl group, (5-substituted-2-oxo-1,3-dioxol-4-yl)methyl (for example (5-methyl-2-oxo-1,3-dioxol-4-yl)methyl), and the like.

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compositions of the present invention are also useful for reducing the risk of secondary outcomes of obesity, such as reducing the risk of left ventricular hypertrophy.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

The terms “administration of” and or “administering a” compound should be understood to mean providing a compound of the invention or a prodrug of a compound of the invention to a subject in need of treatment. The instant pharmaceutical compositions include administration of a single pharmaceutical dosage formulation which contains an appetite suppressant and/or a metabolic rate enhancer and/or a nutrient absorption inhibitor, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the appetite suppressant and/or the metabolic rate enhancer and/or the nutrient absorption inhibitor is realized by the patient at substantially the same time. Such beneficial effect is preferably achieved when the target blood level concentrations of each active drug are maintained at substantially the same time. It is preferred that the combination of the appetite suppressant and/or the metabolic rate enhancer and/or the nutrient absorption inhibitor be co-administered concurrently on a once-a-day dosing schedule; however, varying dosing schedules, such as the appetite suppressant once a day and the nutrient absorption inhibitor once, twice or more times per day, is also encompassed herein. A single oral dosage formulation comprised of both agents in the combination, for example appetite suppressant and a metabolic rate enhancer, is preferred. A single dosage formulation will provide convenience for the patient, which is an important consideration especially for patients with diabetes or obese patients who may be in need of multiple medications.

The term “subject”, as used herein refers to an animal, preferably a mammal, most preferably a human, who has been the object of treatment, observation or experiment. In one embodiment the term “mammal” is a “human” said human being either male or female. The instant combinations are also useful for treating or preventing obesity and obesity-related disorders in cats and dogs. As such, the term “mammal” includes companion animals such as cats and dogs.

The term “subject in need thereof” refers to a subject who is in need of treatment or prophylaxis as determined by a researcher, veterinarian, medical doctor or other clinician. In one embodiment, a subject in need of treatment is an obese human with or without obesity-related co-morbidities.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity or an obesity-related disorder in subjects as risk for obesity or the obesity-related disorder.

Compounds with “different biological mechanisms of action” as used herein are compounds that work based on different physiological modes of action. For example NPY1 antagonists and Mc4r (melanocortin-4 receptor) agonists have different biological mechanisms of action because they work based on different physiological modes of action, namely antagonism of the NPY1 receptor and agonism of the melanocortin-4 receptor. NPY1 antagonists, in general, work primarily based on the same physiological mode of action, antagonism of the NPY1 receptor, therefore all NPY 1 antagonists are considered to have the same biological mechanism.

The magnitude of prophylactic or therapeutic dose of the active ingredients (e.g. the appetite suppressant, and/or the metabolic rate enhancer, and/or the nutrient absorption inhibitor) of the composition will, of course, vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

For use where a composition for intravenous administration is employed, a suitable dosage range is from about 0.0001 mg/kg to about 50 mg/kg, preferably from 0.001 mg/kg to about 20 mg/kg of each compound in the composition per day.

In the case where an oral composition is employed, a suitable dosage range is, e.g. from about 0.001 mg/kg to about 100 mg/kg of each compound in the composition per day, preferably from about 0.01 mg to about 1000 mg per day. For oral administration, the compositions are preferably provided in the form of tablets containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating obesity and/or an obesity related disorder, the agents in the combination (e.g. the appetite suppressant, and/or the metabolic rate enhancer, and/or the nutrient absorption inhibitor) are administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form.

Leptin may be administered at a daily dosage of from about 0.01 mg/kg to about 20 mg/kg, preferably, from about 0.01 mg/kg to about 0.3 mg/kg, preferably injected in a single dose or in divided doses.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of the agents in the combinations of the present invention (e.g. the appetite suppressant; the metabolic rate enhancer; the nutrient absorption inhibitor) may be varied and will depend upon the effective dose of each ingredient. Generally, an effective dose of each will be used. Thus, for example, when an appetite suppressant, such as an NPY1 antagonist, is combined with a metabolic rate enhancer, such as a β3 agonist, the weight ratio of the NPY 1 antagonist to the β3 agonist will generally range from about 1000:1 to about 1:1000, preferably about 200:1 to about 1:200. Compositions of the agents in the combinations of the present invention (e.g. the appetite suppressant; the metabolic rate enhancer; the nutrient absorption inhibitor) will generally also be within the aforementioned range, but in each case, an effective dose of each active ingredient should be used.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an appetite suppressant and/or a metabolic rate enhancer and/or a nutrient absorption inhibitor, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of one or more appetite suppressants, and/or one or more metabolic rate enhancers, and/or one or more nutrient absorption inhibitors, as active ingredients or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. each appetite suppressant, each metabolic rate enhancer, each nutrient absorption inhibitor) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g. each appetite suppressant, each metabolic rate enhancer, and each nutrient absorption inhibitor) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (eg. each appetite suppressant, each metabolic rate enhancer, and each nutrient absorption inhibitor) for the symptomatic adjustment of the dosage to the subject to be treated.

Exemplifying the invention is a pharmaceutical composition comprising an appetite suppressant and a metabolic rate enhancer described above and a pharmaceutically acceptable carrier. Further exemplifying the invention is a pharmaceutical composition comprising an appetite suppressant and a nutrient absorption inhibitor described above and a pharmaceutically acceptable carrier. Further exemplifying the invention is a pharmaceutical composition comprising a metabolic rate enhancer and a nutrient absorption inhibitor described above and a pharmaceutically acceptable carrier. Further exemplifying the invention is a pharmaceutical composition comprising two appetite suppressants described above and a pharmaceutically acceptable carrier. Further exemplifying the invention is a pharmaceutical composition comprising two metabolic rate enhancers described above and a pharmaceutically acceptable carrier. Further exemplifying the invention is a pharmaceutical composition comprising two nutrient absorption inhibitors described above and a pharmaceutically acceptable carrier.

Also exemplifying the invention is a pharmaceutical composition made by combining any of the appetite suppressants and/or metabolic rate enhancers and/or nutrient absorption inhibitors described above and a pharmaceutically acceptable carrier. An illustration of the invention is a process for making a pharmaceutical composition comprising combining any of the appetite suppressants and/or metabolic rate enhancers and/or nutrient absorption inhibitors described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Injectable Suspension (I.M.) | mg/mL |
|---|---|
| CB-1 antagonist (Rimonabant) | 0.70 |
| Nalmefene | 1.0 |
| cyclodextrin | Q.S. ed to |
| (35% weight/volume) | 1 ml volume |
| glycerol | 63.05 |
| Water for injection to a total volume of | 1 mL |
| Tablet | mg/tablet |
| CB-1 antagonist (Rimonabant) | 25 |
| Orlistat | 20 |
| Microcrystalline Cellulose | 40.5 |
| Lactose | 101.5 |
| Croscarmellose Sodium | 5.0 |
| Hydroxypropylcellulose | 6.0 |
| Sodium Dodecyl Sulfate | 1.0 |
| Magnesium Stearate | 1.0 |
| | 200 |
| Capsule | mg/capsule |
| CB-1 antagonist (Rimonabant) | 100 |
| Orlistat | 20 |
| Lactose | 65 |
| Sodium Dodecyl Sulfate | 15 |
| | 200 |
| Aerosol | Per canister |
| CB-1 antagonist (Rimonabant) | 4 mg |
| Orlistat | 9 mg |
| Lecithin, NF Liq. Conc. | 1.2 mg |
| Trichlorofluoromethane, NF | 4.025 g |
| Dichlorodifluoromethane, NF | 12.15 g |

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

### Melanocortin Receptor Agonist—MCH1R Knock Out Mouse Study

### Materials and Methods

Wild type or MCH1R KO mice were implanted with intracerebroventricular (icv) cannulae for icv administration of compounds. One microgram of MT-II was given by icv injection per animal at the onset of dark cycle and food intake was measured 2 hours post-injection. Overnight body weight differences were also measured. Artificial cerebrospinal fluid (aCSF) was used as a vehicle to dissolve MT-II.

### Results

FIG. 1 shows that MT-II significantly inhibited the 2 hour food intake in both wild type and MCH1R KO mice. FIG. 2 shows that MT-II treated mice showed a reduced body weight compared to vehicle treated mice in both wild type and MCH1R KO mice.

### EXAMPLE 2

### CB-1 Inverse Agonist—NPY Knock Out Mouse Study

### Materials and Methods

AM25 1 was given to wild type, NPY KO Mice, and AGRP-NPY double KO mice orally at 3 mg/kg. Overnight food intake was measured.

### Results

FIG. 3 shows AM251, a CB1R inverse agonist, significantly inhibits the food intake in wild type, AGRP-NPY double KO mice and NPY KO mice.

### EXAMPLE 3

### CB-1 Inverse Agonist—MCH1R Knock Out Mouse Study

### Materials and Methods

AM251 was given to wild type and MCH1R KO mice orally at 5 mg/kg. Two hour and overnight food intake were measured.

### Results

FIG. 4 shows that AM 251 (5 mg/kg) significantly inhibited the 2 hour and overnight food intake in both wild type and MCH1R KO mice.

Representative examples of 11β-HSD-1 inhibitors that can be used in the present invention can be synthesized in accordance with the following examples:

### EXAMPLE 4

### 3-[(3,5,7-trimethyl-1-adamantyl)methyl]-6,7,8,9-tetrahydro-5H-[1,2,4]triazolo[4,3-a]azepine trifluoroacetate salt

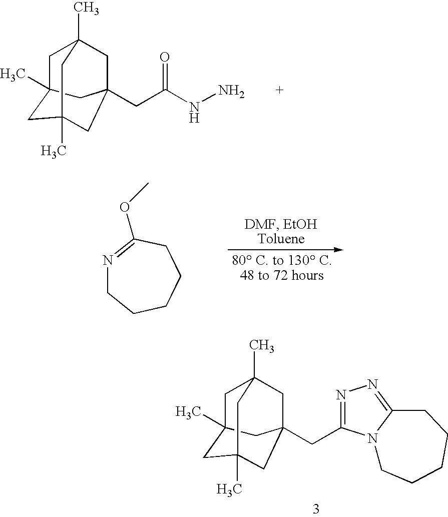

The referenced compound was synthesized as part of a 2-D, single, pure compound library using a Myriad Core System. All reaction vessels were dried under a stream of nitrogen at 120° C. for 12 hours prior to use. All solvents were dried over sieves for at least 12 hours prior to use. All subunits (imino ethers and acyl hydrazides) were dissolved in appropriate solvents immediately prior to use. The following table details the amounts of the subunits and solvents used in the preparation of the library:

| | | | | | equiva- |
|---|---|---|---|---|---|
| Substance | Amount | MW | Concentration | Mmoles | lents |
| Anhydrous | 2.8 mL | N/A | N/A | N/A | N/A |
| Ethanol | | | | | |
| X-axis | 0.48 mL | N/A | 0.25 M in | 0.12 | 1.2 |
| Iminoether | | | Anhydrous | | |
| | | | Ethanol | | |
| Y-axis | 0.71 mL | N/A | 0.14 M in 2.5:1 | 0.10 | 1.0 |
| Hydrazide | | | DMF: EtOH | | |
| Toluene | 3 to 4 mL | N/A | N/A | N/A | N/A |

To 10 mL fritted Myriad reaction vessels under nitrogen was added 2.8 ml of anhydrous ethanol. To each of the reaction vessels was then added an ethanolic solution of the X-component imino ethers (0.48 ml, 0.12 mmoles, 0.25 M in ethanol). Next, was added the appropriate Y-component hydrazide (0.71 mL, 0.1 mmoles, 0.14 M in 2.5:1 DMF:Ethanol). The reactions were aged for 1 hour at room temperature followed by 48 hours at 80° C., after which they were cooled to room temperature. Throughout the incubation, the reactions were gas agitated (1 second pulse of nitrogen every 1 hour.) Once cooled to room temperature, the crude reaction mixtures were analyzed by LC-MS (Method 1). Analysis by LC-MS indicated that the reactions were complete.

The crude reaction was purified by preparative HPLC using mass based detection (Method 2). The collected fractions were analyzed for purity by LC-MS (Method 3); fractions found to be greater than 90% pure were pooled into tared 40 mL EPA vials and lyophilized.

### HPLC Purification Conditions

Analytical LC Method 1:

| | |
|---|---|
| Column: | MetaChem Polaris C-18A, 30 mm × 4.6 mm, 5.0 μm |
| Eluent A: | 0.1% TFA in Water |
| Eluent B: | 0.1% TFA in Acetonitrile |
| Gradient: | 5% B to 95% B in 3.3 minutes, ramp back to 5% B in 0.3 min |
| Flow: | 2.5 mL/min. |
| Column Temperature: | 50° C. |
| Injection amount: | 5 Tl of undiluted crude reaction mixture. |
| Detection: | UV at 220 and 254 nm. |
| | MS: API-ES ionization mode, mass scan range (100-600) |
| | ELSD: Light Scattering Detector |

Preparative LC Method 2:

| | |
|---|---|
| Column: | MetaChem Polaris C-18A, 100 mm × 21.2 mm, 10 μm |
| Eluent A: | 0.1% TFA in Water |
| Eluent B: | 0.1% TFA in Acetonitrile |
| Pre-inject Equilibration: | 1.0 min |
| Post-Inject Hold: | 1.0 min |
| Gradient: | 10% B to 100% B in 6.0 minutes, hold at 100% B for an |
| | additional 2.0 minutes, ramp back from 100% B o 10% B in 1.5 |
| | minutes. |
| Flow: | 20 mL/min. |
| Column Temperature: | ambient |
| Injection amount: | 1.5 ml of undiluted crude reaction mixture. |
| Detection: | MS: API-ES ionization mode, mass scan range (100-600), fraction |
| | collection triggered by detection of M + 1 |

Analytical LC Method 3:

| | |
|---|---|
| Column: | MetaChem Polaris C-18A, 30 mm × 2.0 mm, 3.0 μm |
| Eluent A: | 0.1% TFA in Water |
| Eluent B: | 0.1% TFA in Acetonitrile |
| Gradient: | 5% B to 95% B in 2.0 minutes, ramp back to 5% B in 0.1 min |
| Flow: | 1.75 mL/min. |
| Column Temperature: | 60° C. |
| Injection amount: | 5 Tl of undiluted fraction. |
| Detection: | UV at 220 and 254 nm. |
| | MS: API-ES ionization mode, mass scan range (100-600) |
| | ELSD: Light Scattering Detector |

### Lyophilization Parameters

Initial Freeze Setpoint: 1 hour at −70° C. Drying Phase Condenser Setpoint: −50° C.

Drying Phase Table:

| | | Vacuum |
|---|---|---|
| Shelf Temperature (° C.) | Duration (minutes) | Setpoint (mTorr) |
| −60 | 240 | 25 |
| −40 | 240 | 25 |
| 5 | 480 | 25 |
| 20 | 1000 | 25 |

Retention time: 2.048 min. ESI (m/z): 327.0

### EXAMPLE 5

### 3-(1-adamantyl)-4-ethyl-5-(ethylthio)-4H-1,2,4-triazole

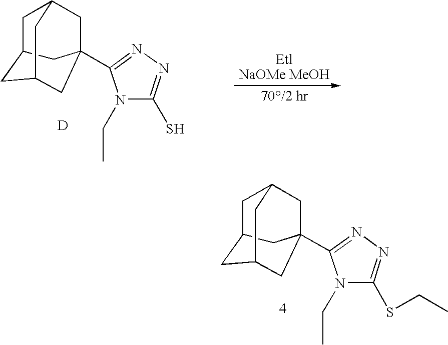

5-(1-Adamantyl)-4-ethyl-4H-1,2,4-triazole-3-thiol (D, Arzneim. -Forsch. 1991, 41, 1260-1264) (40 mg, 0.15 mmoles) and 0.5 M methanolic NaOMe (0.3 ml, 0.15 mmoles) in methanol (1 ml) was heated under reflux for 10 min. Ethyl iodide (12 μl, 0.15 mmoles) was added, and the mixture was heated under reflux for 2 hr. The methanol was removed in vacuo, and the residue was partitioned between CH₂Cl₂ and water. The organic layer was dried (MgSO₄) and evaporated in vacuo. The residue was purified by chromatography on silica gel with 10% MeOH in CH₂Cl₂ to give 3-(1-adamantyl)-4-ethyl-5-(ethylthio)4H-1,2,4-triazole (4),

MS: 278 (M+1). MS ESI (m/z) 292.

### EXAMPLE 6

### 3-(1-adamantyl)-5-(3,4,5-trimethoxyphenyl)-4-methyl-4H-1,2,4-triazole

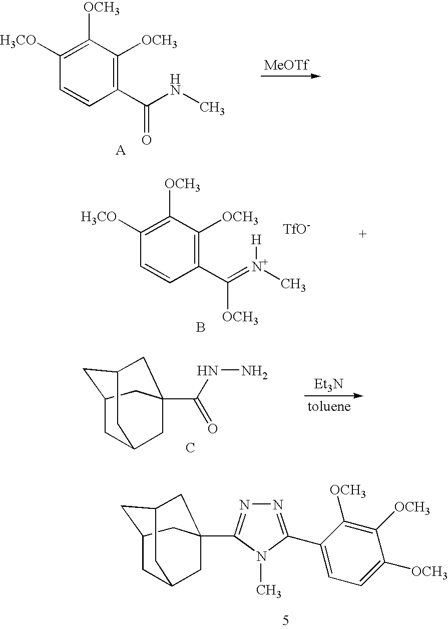

A mixture of compound (A) (16.6 mmoles) and a molar excess of methyl trifluoromethanesulfonate were reacted in a nitrogen atmosphere until the the imino ether triflate salt (B) was obtained, as confirmed by NMR. Excess methyl trifluoromethane sulfonate was removed in vacuo. Toluene (26 ml), triethylamine (3.86 ml, 27.7 mmoles) and adamantane-1-carbohydrazide (C) (2.15 g, 11.1 mmoles) were added, and the mixture was stirred at 65° C. for 5 hrs. The mixture was heated at 110° for 3 hr. The cooled reaction was diluted with ethyl acetate (75 ml), washed with water (75 ml) and saturated brine (30 ml), and dried (MgSO₄). The ethyl acetate was evaporated in vacuo. Elution with 7% methanol in chloroform and evaporation in vacuo gave compound 5. Recrystallization from isopropyl ether affords pure 5. MS: ESI (m/z) 384.

N-methyl amide starting material that is not available commercially is prepared by EDC/DMAP mediated reaction between the appropriate methyl ester or the acid chloride reacted at room temperature with 40% aqueous methylamine.

### EXAMPLE 7

### 3-adamantanyl-4,5,6,7,8,9,10,11,12,3a-decahydro-1,2,4-triazolo[4,3-a][11]annulene

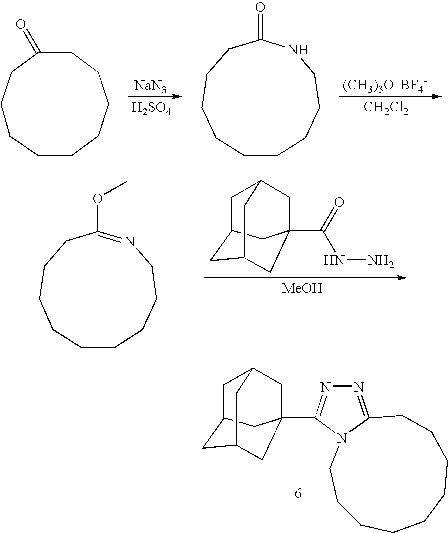

Cyclodecanone (1.0 g) in 10 mL concentrated sulfuric acid was cooled to 0° C. and 0.54 g of sodium azide was added. The reaction continued to stir at 0° C. for 1 hour and warmed to room temperature where it was stirred for two hours. The solution was diluted with cold water and treated with cold 10% NaOH solution until pH=9. Extraction with ether (2×), drying over magnesium sulfate and evaporation of solvent provided 1.23 g of 2-azacycloundecanone.

2-azacycloundecanone (0.87 g) was dissolved in 20 mL methylene chloride and stirred at room temperature under nitrogen. 1.5 g trimethyloxonium tetrafluoroborate was added and the reaction stirred overnight. The mixture was added to saturated aqueous sodium bicarbonate and extracted with methylene chloride (2×). The combined organic layers were washed with brine, dried over magnesium sulfate, and the solvent evaporated to provide crude 2-methoxyazacyclododec-1-ene.

Adamantanecarbohydrazide (45 mg) was added to a small dry flask and dissolved in 3 mL dry methanol. 63.7 mg of 2-methoxyazacyclododec-1-ene was added and the mixture was refluxed at 70° C. overnight. The methanol was removed by evaporation and 3 mL toluene added. This mixture was refluxed 24 hours at 122° C. The toluene was evaporated and the resulting solid was purified by preparative HPLC. (100% gradient/12min) to provide 6 as the trifluoroacetate salt.

11β-HSD1 inhibition can be demonstrated in accordance with the following assay:

### 11β-HSD 1 Inhibition Constants

In vitro enzymatic activity is assessed for test compounds via a Scintillation Proximity Assay (SPA). Tritiated-cortisone substrate, NADPH cofactor and titrated compound are incubated with 11β-HSD1 enzyme at 37° C. to allow conversion to cortisol to progress. Following this incubation, protein A coated SPA beads, pre-blended with anti-cortisol monoclonal antibody and a non-specific 11β-HSD inhibitor, are added to each well. The mixture is shaken at 15° C. and then read on a liquid scintillation counter suitable for 96 well plates.

Percent inhibition is calculated relative to a non-inhibited control well, and IC₅₀ curves are generated.

For ultimate calculation of percent inhibition, a series of wells is added that represent the assay minimum and maximum: one set that contains substrate without compound or enzyme (background), and another set that contains substrate and enzyme without any compound (maximum signal). The plates are spun briefly at a low speed in a centrifuge to pool the reagents, sealed with an adhesive strip, mixed gently, and incubated at 37° C. for 2 hours. After incubation, 45 TL of SPA beads, pre-suspended with anti-cortisol monoclonal antibody and non-specific 11β-HSD inhibitor, are added to each well. The plates are resealed and shaken gently for greater than 1.5 hours at 15° C. Data is collected on a plate based liquid scintillation counter such as a Topcount. To control for inhibition of anti-cortisol antibody/cortisol binding, substrate spiked with 1.25 nM [3]H cortisol is added to designated single wells. 1 TL of 200 μM compound is added to each of these wells, along with 10 TL of buffer instead of enzyme. Any calculated inhibition is due to compound interfering with the cortisol binding to the antibody on the SPA beads.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A composition comprising two appetite suppressants, wherein each appetite suppressant is selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin agonist/modulator;
(14) a leptin derivative;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) a 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof;
provided that when the first appetite suppressant is a NPY1 antagonist, then the second appetite suppressant is not selected from the group consisting of: a MCH1R antagonist, a MCH2R antagonist, leptin, a leptin derivative, 5HT2c agonist, a Mc4r agonist, a serotonin reuptake inhibitor, and a GLP-1 agonist;
provided that when the first appetite suppressant is leptin, then the second appetite suppressant is not selected from the group consisting of: a MCH-1R antagonist, a MCH-2R antagonist, a NPY1 antagonist, a leptin derivative, 5HT2c agonist, a Mc4r agonist, a serotonin reuptake inhibitor, a GLP-1 agonist, a CCK-A agonist, an opioid antagonist, and a monoamine reuptake inhibitor;
provided that when the first appetite suppressant is a CB-1 antagonist/inverse agonist, then the second appetite suppressant is not selected from the group consisting of: an opioid antagonist, a serotonin reuptake inhibitor, and a monoamine reuptake inhibitor; provided that when the first appetite suppressant is an opioid antagonist, then the second appetite suppressant is not a serotonin reuptake inhibitor; and
provided that the appetite suppressants have different biological mechanisms of action.

2. The composition of claim 1 wherein the appetite suppressant is selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) an opioid antagonist;
(13) an orexin antagonist;
(14) a BRS3 agonist;
(15) a CCK-A agonist;
(16) CNTF;
(17) a CNTF agonist/modulator;
(18) a CNTF derivative;
(19) a 5HT2c agonist;
(20) a Mc4r agonist;
(21) a monoamine reuptake inhibitor;
(22) a serotonin reuptake inhibitor;
(23) a GLP-1 agonist;
(24) axokine;
(25) fenfluramine;
(26) nalmafene;
(27) phentermine;
(28) rimonabant;
(29) sibutramine; and
(30) topiramate;
and pharmaceutically acceptable salts and esters thereof;
provided that when the first appetite suppressant is a NPY1 antagonist, then the second appetite suppressant is not selected from the group consisting of: a MCH1R antagonist, a MCH2R antagonist, 5HT2c agonist, a Mc4r agonist, a serotonin reuptake inhibitor, and a GLP-1 agonist;
provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor;
provided that when the first appetite suppressant is an opioid antagonist, then the second appetite suppressant is not a serotonin reuptake inhibitor; and
provided that the appetite suppressants have different biological mechanisms of action.

3. The composition of claim 2 wherein the first appetite suppressant is a Mc4r agonist, and pharmaceutically acceptable salts and esters thereof, and the second appetite suppressant is selected from the group consisting of
(1) a MCH1R antagonist; and
(2) a MCH2R agonist/antagonist;
and pharmaceutically acceptable salts and esters thereof.

4. The composition of claim 2 wherein the first appetite suppressant is a CB-1 antagonist/inverse agonist, and pharmaceutically acceptable salts and esters thereof, and the second appetite suppressant is selected from the group consisting of
(1) a NPY1 antagonist;
(2) a NPY2 agonist;
(3) a NPY4 agonist;
(4) a MCH1R antagonist;
(5) a MCH2R agonist/antagonist; and
(6) a Mc4r agonist;
and pharmaceutically acceptable salts and esters thereof.

5. The composition of claim 1 further comprising a pharmaceutically acceptable carrier.

6. A method of treating a subject having a disorder associated with excessive food intake comprising administration of a therapeutically effective amount of two appetite suppressants selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin agonist/modulator;
(14) a leptin derivative;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) a 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof;
to a subject in need of such treatment;
provided that when the first appetite suppressant is a NPY1 antagonist, then the second appetite suppressant is not selected from the group consisting of: a MCH1R antagonist, a MCH2R antagonist, leptin, a leptin derivative, 5HT2c agonist, a Mc4r agonist, a serotonin reuptake inhibitor, and a GLP-1 agonist;
provided that when the first appetite suppressant is leptin, then the second appetite suppressant is not selected from the group consisting of: a MCH-1R antagonist, a MCH-2R antagonist, a NPY1 antagonist, a leptin derivative, 5HT2c agonist, a Mc4r agonist, a serotonin reuptake inhibitor, a GLP-1 agonist, a CCK-A agonist, an opioid antagonist, and a monoamine reuptake inhibitor;
provided that when the first appetite suppressant is a CB-1 antagonist/inverse agonist, then the second appetite suppressant is not selected from the group consisting of an opioid antagonist, a serotonin reuptake inhibitor, and a monoamine reuptake inhibitor; and provided that the appetite suppressants have different biological mechanisms of action.

7. The method according to claim 6 wherein the disorder associated with excessive food intake is obesity.

8. The method according to claim 7 wherein the disorder associated with excessive food intake is an obesity-related disorder.

9. The method according to claim 8 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Turner's syndrome; metabolic syndrome; and acute lymphoblastic leukemia.

10. The method according to claim 9 wherein the obesity-related disorder is diabetes.

11. A composition comprising
(a) an appetite suppressant selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin derivative;
(14) a leptin agonist/modulator;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof; and
(b) a metabolic rate enhancer selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone, agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)-4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof;
provided that when the metabolic rate enhancer is a β3 agonist, then the appetite suppressant is not selected from the group consisting of: a CB-1 antagonist/inverse agonist, a MCH1R antagonist, a MCH2R antagonist, leptin, a leptin derivative, a CCK-A agonist, a 5HT2c agonist, a Mc4r agonist, a monoamine reuptake inhibitor, a serotonin reuptake inhibitor, and a GLP-1 agonist;
provided that when the metabolic rate enhancer is a UCP-1,2 or 3 activator, then the appetite suppressant is not selected from the group consisting of: leptin, and a leptin derivative;
provided that when the metabolic rate enhancer is an 11β HSD-1 inhibitor, then the appetite suppressant is not selected from the group consisting of: a CB-1 antagonist/inverse agonist, a Mc4r agonist, a monoamine reuptake inhibitor, and a serotonin reuptake inhibitor; and
provided that when the appetite suppressant is a monoamine reuptake inhibitor, then the metabolic rate enhancer is not a PDE inhibitor.

12. A composition comprising an appetite suppressant selected from the group consisting of: a NPY5 antagonist, and pharmaceutically acceptable salts and esters thereof; and metabolic rate enhancer selected from the group consisting of: an 11β HSD-1 inhibitor, and pharmaceutically acceptable salts and esters there.

13. The composition of claim 12 further comprising a pharmaceutically acceptable carrier.

14. A method of treating a subject having a disorder associated with excessive food intake comprising administration of
(a) a therapeutically effective amount of an appetite suppressant selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin agonist/modulator;
(14) a leptin derivative;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof; and
(b) a therapeutically effective amount of a metabolic rate enhancer selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone β agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)-4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof;
to a subject in need of such treatment;
provided that when the metabolic rate enhancer is a β3 agonist, then the appetite suppressant is not selected from the group consisting of: a CB-1 antagonist/inverse agonist, a MCH1R antagonist, a MCH2R antagonist, leptin, a leptin derivative, a CCK-A agonist, a 5HT2c agonist, a Mc4r agonist, a monoamine reuptake inhibitor, a serotonin reuptake inhibitor, and a GLP-1 agonist;
provided that when the metabolic rate enhancer is a UCP-1, 2 or 3 activator, then the appetite suppressant is not selected from the group consisting of: leptin, and a leptin derivative;
provided that when the metabolic rate enhancer is an 11β HSD-1 inhibitor, then the appetite suppressant is not selected from the group consisting of: a CB-1 antagonist/inverse agonist, a Mc4r agonist, a monoamine reuptake inhibitor, and a serotonin reuptake inhibitor; and
provided that when the appetite suppressant is a monoamine reuptake inhibitor, then the metabolic rate enhancer is not a PDE inhibitor.

15. The method according to claim 14 wherein the disorder associated with excessive food intake is obesity.

16. The method according to claim 15 wherein the disorder associated with excessive food intake is an obesity-related disorder.

17. The method according to claim 16 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Turner's syndrome; metabolic syndrome; and acute lymphoblastic leukemia.

18. The method according to claim 17 wherein the obesity-related disorder is diabetes.

19. A composition comprising
(a) an appetite suppressant selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin agonist/modulator;
(14) a leptin derivative;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) a 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof; and
(b) a nutrient absorption inhibitor selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof;
provided that when the appetite suppressant is a monoamine reuptake inhibitor, then the nutrient absorption inhibitor is not a lipase inhibitor.

20. The composition of claim 19 further comprising a pharmaceutically acceptable carrier.

21. A method of treating a subject having a disorder associated with excessive food intake comprising administration of
(a) a therapeutically effective amount of an appetite suppressant selected from the group consisting of
(1) a 5HT transporter inhibitor;
(2) a NE transporter inhibitor;
(3) a CB-1 antagonist/inverse agonist;
(4) a ghrelin antagonist;
(5) a H3 antagonist/inverse agonist;
(6) a MCH1R antagonist;
(7) a MCH2R agonist/antagonist;
(8) a NPY1 antagonist;
(9) a NPY2 agonist;
(10) a NPY4 agonist;
(11) a mGluR5 antagonist;
(12) leptin;
(13) a leptin agonist/modulator;
(14) a leptin derivative;
(15) an opioid antagonist;
(16) an orexin antagonist;
(17) a BRS3 agonist;
(18) a CCK-A agonist;
(19) CNTF;
(20) a CNTF agonist/modulator;
(21) a CNTF derivative;
(22) a 5HT2c agonist;
(23) a Mc4r agonist;
(24) a monoamine reuptake inhibitor;
(25) a serotonin reuptake inhibitor;
(26) a GLP-1 agonist;
(27) axokine;
(28) fenfluramine;
(29) nalmafene;
(30) phentermine;
(31) rimonabant;
(32) sibutramine;
(33) topiramate; and
(34) phytopharm compound 57;
and pharmaceutically acceptable salts and esters thereof; and
(b) a therapeutically effective amount of a nutrient absorption inhibitor selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof;
to a subject in need of such treatment;
provided that when the appetite suppressant is a monoamine reuptake inhibitor, then the nutrient absorption inhibitor is not a lipase inhibitor.

22. The method according to claim 21 wherein the disorder associated with excessive food intake is obesity.

23. The method according to claim 22 wherein the disorder associated with excessive food intake is an obesity-related disorder.

24. The method according to claim 23 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Turner's syndrome; metabolic syndrome; and acute lymphoblastic leukemia.

25. The method according to claim 24 wherein the obesity-related disorder is diabetes.

26. A composition comprising two metabolic rate enhancers, wherein each metabolic rate enhancer is selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone β agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof;
provided that when the first metabolic rate enhancer is an 11β HSD-1 inhibitor, then the second metabolic rate enhancer is not a β3 agonist; and provided that the metabolic rate enhancers have different biological mechanisms of action.

27. The composition of claim 26 further comprising a pharmaceutically acceptable carrier.

28. A method of treating a subject having a disorder associated with excessive food intake comprising administration of a therapeutically effective amount of two metabolic rate enhancers selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone β agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)-4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof;
to a subject in need of such treatment;
provided that when the first metabolic rate enhancer is an 11β HSD-1 inhibitor, then the second metabolic rate enhancer is not a β3 agonist; and
provided that the metabolic rate enhancers have different biological mechanisms of action.

29. The method according to claim 28 wherein the disorder associated with excessive food intake is obesity.

30. The method according to claim 29 wherein the disorder associated with excessive food intake is an obesity-related disorder.

31. The method according to claim 30 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Tumer's syndrome; metabolic syndrome; and acute lymphoblastic leukemia.

32. The method according to claim 31 wherein the obesity-related disorder is diabetes.

33. A composition comprising a metabolic rate enhancer, and pharmaceutically acceptable salts and esters thereof, and a nutrient absorption inhibitor, and pharmaceutically acceptable salts and esters thereof.

34. The composition of claim 33 comprising
(a) a metabolic rate enhancer selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone β agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)-4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof; and
(b) a nutrient absorption inhibitor selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof.

35. The composition of claim 34 further comprising a pharmaceutically acceptable carrier.

36. A method of treating a subject having a disorder associated with excessive food intake comprising administration of
(a) a therapeutically effective amount of a metabolic rate enhancer selected from the group consisting of
(1) an ACC2 inhibitor;
(2) a β3 agonist;
(3) a DGAT1 inhibitor;
(4) a DGAT2 inhibitor;
(5) a FAS inhibitor;
(6) a PDE inhibitor;
(7) a thyroid hormone β agonist;
(8) an UCP-1, 2, or 3 activator;
(9) an acyl-estrogen;
(10) a glucocorticoid antagonist;
(11) an 11β HSD-1 inhibitor;
(12) a Mc3r agonist;
(13) a SCD-1;
(14) oleoyl-estrone;
(15) 3-[(3,5,7-trimethyl-1-adamantyl)methyl]-6,7,8,9-tetrahydro-5H-[1,2,4]triazolo[4,3-a]azepine;
(16) 3-(1-adamantyl)-4-ethyl-5-(ethylthio)-4H-1,2,4-triazole;
(17) 3-adamantanyl-4,5,6,7,8,9,10,11,12,3a-decahydro-1,2,4-triazolo[4,3-a][11]annulene; and
(18) 3-(1-adamantyl)-5-(3,4,5-trimethoxyphenyl)-4-methyl-4H-1,2,4-triazole;
and pharmaceutically acceptable salts and esters thereof; and
(b) a therapeutically effective amount of a nutrient absorption inhibitor selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof;
to a subject in need of such treatment.

37. The method according to claim 36 wherein the disorder associated with excessive food intake is obesity.

38. The method according to claim 37 wherein the disorder associated with excessive food intake is an obesity-related disorder.

39. The method according to claim 38 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Turner's syndrome; metabolic syndrome; and acute lymphoblastic leukemia.

40. The method according to claim 39 wherein the obesity-related disorder is diabetes.

41. A composition comprising two nutrient absorption inhibitors, and pharmaceutically acceptable salts and esters thereof, provided that the nutrient absorption inhibitors have different biological mechanisms of action.

42. The composition of claim 41, wherein each nutrient absorption inhibitor is selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof;
provided that the nutrient absorption inhibitors have different biological mechanisms of action.

43. The composition of claim 42 further comprising a pharmaceutically acceptable carrier.

44. A method of treating a subject having a disorder associated with excessive food intake comprising administration of a therapeutically effective amount of two nutrient absorption inhibitors selected from the group consisting of
(1) a lipase inhibitor;
(2) a fatty acid transporter inhibitor;
(3) a dicarboxylate transporter inhibitor;
(4) a glucose transporter inhibitor;
(5) a phosphate transporter inhibitor; and
(6) orlistat;
and pharmaceutically acceptable salts and esters thereof;
to a subject in need of such treatment;
provided that the nutrient absorption inhibitors have different biological mechanisms of action.

45. The method according to claim 44 wherein the disorder associated with excessive food intake is obesity.

46. The method according to claim 45 wherein the disorder associated with excessive food intake is an obesity-related disorder.

47. The method according to claim 46 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovary disease; craniopharyngioma; the Prader-Willi Syndrome; Frohlich's syndrome; GH-deficient subjects; normal variant short stature; Turner's syndrome; metabolic syndrome; and acute lymphoblastic leukemia.

48. The method according to claim 47 wherein the obesity-related disorder is diabetes.
